SEQUENCE LISTING (19 sequences)

SEQ ID NO: 1 - DNA, 930 bp, Mus musculus
  source 1..930: mol_type = genomic DNA
catggctttctttgaaaactgtgtttagaagatttctgggatttgtgtgcaaaagcaccttgttggccctcaccgtgacgttttagggaagacttcccatctctcaaggtgggaaggcttggaggtggtgtcttgtggcctcctatggtggttaggtacttctcagaagacaggactggaaattagataatgtctgatgtcatatcattcacaataccaaaaaaaccctggtgtcccgatggctataaaagcagcaacttctgcctctcccatcacaagcagagacacctaaacaggctctcctctcagttatcaactgttgacacttgtgcgatctctgatggctgtcctgcagaaatctatgagtttttcccttatggggactttggccgccagctgcctgcttctcattgccctgtgggcccaggaggcaaatgcgctgcccgtcaacacccggtgcaagcttgaggtgtccaacttccagcagccatacatcgtcaaccgcacctttatgctggccaaggaggccagccttgcagataacaacacagatgtccggctcatcggggagaaactgttccgaggagtcagtgctaaggatcagtgctacctgatgaagcaggtgctcaacttcaccctggaagacgttctgctcccccagtcagacaggttccagccctacatgcaggaggtggtgcctttcctgaccaaactcagcaatcagctcagctcctgtcacatcagcggtgacgaccagaacatccagaagaatgtcagaaggctgaaggagacagtgaaaaagcttggagagagtggagagatcaaggcgattggggaactggacctgctgtttatgtctctgagaaatgcttgcgtctgagcgagaagaagctagaaaacgaagaactgctccttcctgccttctaaaaa

SEQ ID NO: 2 - DNA, 18 bp, synthetic construct
  source 1..18: mol_type = other DNA
aggctgccccgactacgt

SEQ ID NO: 3 - DNA, 27 bp, synthetic construct
  source 1..27: mol_type = other DNA
gactttctcctggtatgagatagcaaa

SEQ ID NO: 4 - DNA, 20 bp, synthetic construct
  source 1..20: mol_type = other DNA
ttgacggaccccaaaagatg

SEQ ID NO: 5 - DNA, 21 bp, synthetic construct
  source 1..21: mol_type = other DNA
agaaggtgctcatgtcctcat

SEQ ID NO: 6 - DNA, 25 bp, synthetic construct
  source 1..25: mol_type = other DNA
acaagtcggaggcttaattacacat

SEQ ID NO: 7 - DNA, 22 bp, synthetic construct
  source 1..22: mol_type = other DNA
ttgccattgcacaactcttttc

SEQ ID NO: 8 - DNA, 20 bp, synthetic construct
  source 1..20: mol_type = other DNA
ggttgccaagccttatcgga

SEQ ID NO: 9 - DNA, 20 bp, synthetic construct
  source 1..20: mol_type = other DNA
cttctcacccagggaattca

SEQ ID NO: 10 - DNA, 20 bp, synthetic construct
  source 1..20: mol_type = other DNA
gtcaaccgcacctttatgct

SEQ ID NO: 11 - DNA, 20 bp, synthetic construct
  source 1..20: mol_type = other DNA
gttgagcacctgcttcatca

SEQ ID NO: 12 - DNA, 18 bp, synthetic construct
  source 1..18: mol_type = other DNA
tccaggctttgggcatca

SEQ ID NO: 13 - DNA, 25 bp, synthetic construct
  source 1..25: mol_type = other DNA
ctttatcagctgcacatcactcaga

SEQ ID NO: 14-16 - skipped (3 entries)

SEQ ID NO: 17 - protein, 179 aa, Mus musculus
MAVLQKSMSFSLMGTLAASCLLLIALWAQEANALPVNTRCKLEVSNFQQPYIVNRTFMLAKEASLADNNTDVRLIGEKLFRGVSAKDQCYLMKQVLNFTLEDVLLPQSDRFQPYMQEVVPFLTKLSNQLSSCHISGDDQNIQKNVRRLKETVKKLGESGEIKAIGELDLLFMSLRNACV

SEQ ID NO: 18 - DNA, 5363 bp, Homo sapiens
  source 1..5363: mol_type = genomic DNA
acaagcagaatcttcagaacaggtaagcgtttcggcaaacttggtacaattggttagtttgatgaaatacttcttgactaattttgttccttcacgttgtcttcgaccaggttctccttccccagtcaccagttgctcgagttagaattgtctgcaatggccgccctgcagaaatctgtgagctctttccttatggggaccctggccaccagctgcctccttctcttggccctcttggtacagggaggagcagctgcgcccatcagctcccactgcaggcttgacaagtccaacttccagcagccctatatcaccaaccgcaccttcatgctggctaaggaggtatacatctcaatcctgctctttctcgttggatctacttggaatccaaatagttcttaaacttttcttcagagcatctctaagagctttaggaacccactgtttatccctgagggtagataaattttctgttttttcagagactctttgggaatctggcttttttttttttcttgaacttcttccttccattttggcctttatgatacatatgatgaatttttcccaaagagcggccattcagtaatccatctgatgattttttttttcctttatgcctctgtgcattgttctaaactcatgcacacatctgaattctgcttttagtctttatgatgttgctctggggagacgggatggggcacatgtctatgtataaattttttttctatttgctcaatgtccagacccttagtcttttcttctcttccaggctagcttggctgataacaacacagacgttcgtctcattggggagaaactgttccacggagtcagtgtaagctacagttgtgacgaacagggccgtgtgccgtccatgggtacttggggtggtggtgatgatggtttaggtcttatcccttatgaccctttctgtttcccttccacctgcagatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtgtaagttcagctctcagcctatgcccacctacccctccttccctccttccacagagacccccttaccccaactctctctccttccccctacccctaagctagcaggaagaagtgtcttggcagcagtgttatcaggagtcatttgggatcatagagtatttgcttttgctttgactgagtcacatcttgagtttatagtggtgaatggggtctggaacttaagtgtacagaagccgcattggtttgtcttcggaaaaaaggcaactcaggttgcgtaagatgagaaaggtgttgggaaaaacatctagatgtggaaatggatccattgagtctaagttgttgaggggaggggatggcatggagagaaattagaagagaaagtgggaaatgggaaggcttaaagtcggtggtgggtcggcagactgttgccctgttgatgtcatgggaagccacaaaatcggaggcgtgtgaacttgatgccgctgaacatttgaaactatgaaaaaaagtttgagtggagtgggcccagtaaaaggccctaggacttactgaagagggcttaattttcacatgagatgttttatgtacatttcttgttctaagcatgcaattttctggagatacgattgaggttttattccttacagaatttgcataaactactccgctctttccacaaatgcaaacctcagtaggatttcccaaagatgaagagaggtctcttgtaagggaagtgactggattctggcgtccaagggaattcaagagctcaggaaatctaggtcactgttgaaatctaggtcattgtgggcaaaattactaagagctttaattccaggtgaattgtactgtacctccatgggtgtggaggttcataaagtttcagcacaacattaagatagttatgcttgttattgttttatagcatattgaaggtgatgacctgcatatccagaggaatgtgcaaaagctgaaggacacagtgaaaaaggtaggactgataactgtcaatgctaagtcatgcaataggagagacaaatgttgtttttctttcctttctttcttcccatcactttgtgatttttcacttgattctcctaccaccagggcgattactttggtgtctgtgtatgtagatatatctatatatctagatgtcagtttccaaatcttgcaaattgtagaattctagaactggttgggatcttagcttgtctagtcacataacctcagattctggggatggtcagtggcagagatagggctagaatgcaggtctcctgaatcccaagccagcacttttcccggtggtgatacagattagttttggtaccattaattcttagggaaatttcagattcctattgactcatgtaatctgaagaagtacttgtttaaaaacagaaaaatgcctatgggcaaatttatttgaagtcatttttgaagtcattaatgcattgctttgaaacttggaagaataaactcagaacaatgagaaaagagctggacttgcatatagggctaatttctggagtaataaacacttattttgaattatcataatatctatcagatattgattatagtttaaaagcaagagcagacaaccccgatctcttttatacaggttcaaatagagtaaaaatattagtaagagatttattaaagttaaatggaagtctgaattggtaagcttttttttcttcctctctcccatcaagaccttccattctagtttcttccttcactccctcaacaaatccctagggagcatttatccatggtgggctggtgtacatttctatagtgaatgataccatcatgtggcctatttggtgaaaagaacaacaatggaaggcttagactaacaatagtgactcaccccaaaaccggaggaatgattaggagcagtgaaagtgacgctcttgcaagcaggtacaactaaatactcagaaacatgaaggctccagttgatggaattttcagtaacaagcttaaccttaattccccctttttccctcttgactttttaaaaaagcgtttcttcctgagcatcatttaatgagtgtgactgtttcttcctttgataattgaaggctttgtagttttaaattgtgaagcccagttctcttgttatagaactattatctagacatggagggctgaatgttagcatgccacagacaaggcatgctttacacatcttgcttaaaaaattactgatttcatcttgcttgttgtctttagaaaagtgaagtgtgagagaggagaatctcatggtgatctgtgtgattttcaagacctttaatccattttgaaagaatcaatttcatatttgcaatgggttgccatgtggaagagtgattatgcttttttgctggtagcttcagaaagcacaggagggagagcaatgttgttcagagaaagatcaacaggaggagaaactgtcagagctgtctgaaatagggtggttttgggaggcattaattccctctcgttgggggtaaaagcagaacgcaggttggtagtaaaatgcatgacagacagtaggggacgataaactttaaaattctttatagtcttggagtctttgagatagaaaagaatatctttttggccttatgtcaaaagaagtatggaaaggtgaaagggcggaagaaagcaggaaaaggaagaaccatgtattatacagaggacaatggtgacaaggtttttcttgaaataatgcaaatatgatagattagaggaatttcagtagggaatgcttttcacttgaatttgggtttcctcttcgattaagtttgggatcctcatctgcatttgacttggagagagaaagaatgaatgttaggacctatatctggttttctattaactaaagcaagtggaaaagacttatttggtatttttcccacaaaagtgaaaacttttcttttactgtttgtcaaaaaggtggaaatagaaaaagccttaatgtattggtgaatacatggttcaaagtcatttgagtagagatgttttaaatcaggagtgtccaatcatttggcttccctggaccaccttgaaagaattgtcttggtacacacataaaatacaagaacaatagctgatgagctaaaaaagtccatgcataaatctcatactgttttaagaaagtttatgaatttctgttagggtgcattcaaagctgtcctgggccatgtgcggcctgtgggctgcaggttggacaagctccttataagtaatctgtcatagatagttttggagctgcaaaacaggccaaggcataatgggtggcactcgggatcccccagatcccagcctcacttcagtctccttgctctggttaagaaggggtggtcaactctctgcccagcttttaaacagcttcattagtgtgaggtgcacctgaaattgatgcctgctggtggcctctcagtccagagagccgtcattttaagctctttggcaaatcatacaatactaaagggatattactatgaatgttttacaaatgcttaaaactcggtttctgtctccatcaacctaatcttgcaatttctaatttgttcactttagaaaacatggcataaatgctcaaatacttttgcattcttattttcacagcttggagagagtggagagatcaaagcaattggagaactggatttgctgtttatgtctctgagaaatgcctgcatttgaccagagcaaagctgaaaaatgaataactaaccccctttccctgctagaaataacaattagatgccccaaagcgattttttttaaccaaaaggaagatgggaagccaaactccatcatgatgggtggattccaaatgaacccctgcgttagttacaaaggaaaccaatgccacttttgtttataagaccagaaggtagactttctaagcatagatatttattgataacatttcattgtaactggtgttctatacacagaaaacaatttattttttaaataattgtctttttccataaaaaagattactttccattcctttaggggaaaaaacccctaaatagcttcatgtttccataatcagtactttatatttataaatgtatttattattattataagactgcattttatttatatcattttattaatatggatttatttatagaaacatcattcgatattgctacttgagtgtaaggctaatattgatatttatgacaataattatagagctataacatgtttatttgacctcaataaacacttggatatcctaa

SEQ ID NO: 19 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

(19) United States
(12) Patent Application Publication
(10) Pub. No.: US 2026/0041644 A1
(43) Pub. Date: Feb. 12, 2026

(54) LIPID NANOPARTICLES FOR DRUG DELIVERY AND METHODS OF USE THEREOF

(71) Applicant: Georgia State University Research Foundation, Inc., Atlanta, GA (US)

(72) Inventors: Didier Merlin, Atlanta, GA (US); Chunhua Yang, Atlanta, GA (US)

(21) Appl. No.: 19/100,448

(22) PCT Filed: Jul. 31, 2023
(86) PCT No.: PCT/US2023/071336

§ 371 Date: Jan. 31, 2025

Related U.S. Application Data
(60) Provisional application No. 63/369,969, filed on Jul. 31, 2022.

(51) Int. Cl. A61K 9/51; A61K 9/00; A61K 38/20; A61P 1/04

(52) U.S. Cl. CPC A61K 9/5123; A61K 9/0053; A61K 38/20; A61P 1/04

(57) ABSTRACT

Disclosed are compositions, systems, and methods involving lipid nanoparticle primarily composed of phosphatidic acid (PA), monogalactosyldiacylglycerol (MGDG), and digalactosyldiacylglycerol (DGDG). In particular, the PA, MGDG, and DGDG are present in the nanoparticles in useful ratios, preferably falling in a ratio of 3 to 7, I to 3, and 2 to 4, respectively. Further, it is useful for the PA, MGDG, and DGDG to make up 90% or more of the total lipid in the nanoparticles. The disclosed lipid nanoparticles are useful as drug delivery systems for delivery of a drug, such as oral delivery, intravascular delivery, or intramuscular delivery. The disclosed lipid nanoparticles can be used in methods involving administration or delivery of the nanoparticles to a subject. In some forms, the subject can be a disease or condition, such as inflammatory bowel disease, ulcerative colitis, Crohn's disease, cancer, colon cancer, or a coronavirus infection.

### CROSS-REFERENCE TO RELATED APPLICATIONS

This application claims benefit of and priority to U.S. Application No. 63/369,969, filed Jul. 31, 2022, which is incorporated herein by reference in its entirety.

### STATEMENT REGARDING FEDERALLY SPONSORED RESEARCH

This invention was made with government support under Grant No. DK107739 awarded by the National Institutes of Health and Grant No. BX004476 awarded by the Department of Veterans Affairs. The government has certain rights in the invention.

### REFERENCE TO THE SEQUENCE LISTING

The Sequence Listing submitted as a text file named “GSURF_2022_018_02_ST26.xml” created on Jul. 28, 2023, and having a size of 22,350 bytes is hereby incorporated by reference pursuant to 37 C.F.R. § 1.834(c)(1).

### FIELD OF THE INVENTION

The disclosed invention is generally in the field of drug delivery systems and specifically in the area of lipid nanoparticles for drug delivery.

### BACKGROUND OF THE INVENTION

Inflammatory bowel disease (IBD), an umbrella term for Crohn's disease (CD) and ulcerative colitis (UC), is a relapsing and chronic inflammatory disorder of the gastrointestinal (GI) tract that affects more than 3 million adults in the US [1]. The etiology of the disease is still unclear, and there appear to be several non-specific pathophysiological causes, making it challenging to develop effective diagnostic and therapeutic strategies for IBD [2-4]. Recent scientific research has yielded a number of IBD therapeutics, including biologics (e.g., anti-tumor necrosis factor-alpha, anti-interleukin, and anti-integrin), antibiotics, corticosteroids, and immunomodulators.

Unfortunately, most of these treatments are delivered systemically and thus cause severe short- or long-term side effects, such as affecting gut bacterial/fungal function and promoting cancer development [5].

Interleukin-22 (IL-22), a member of the IL-10 cytokine family, is a well-known regulator of epithelial homeostasis. Unique biological properties enable IL-22 to promote wound healing during intestinal inflammation. Moreover, IL-22 plays a protective role against pro-inflammatory mediators and is strongly associated with IBD susceptibility genes, such as jak1, tyk2, and STAT3 [6]. The local microinjection of a lipid/IL-22 cDNA vector complex was shown to activate innate immune pathways in a mouse model of Th2-mediated colitis that closely mimics human UC. Local IL-22 cDNA delivery enhanced STAT3 activation in colonic epithelial cells, induced STAT3-dependent expression of mucus-associated molecules and promoted restitution of goblet cells. Notably, IL-22 cDNA delivery ameliorated local intestinal inflammation, likely via enhanced mucus production. Inhibition of IL-22 activity via overexpression of IL-22-binding protein suppressed goblet cell restitution in the recovery phase of a dextran sulfate sodium (DSS)-induced mouse model of acute colitis. These data collectively indicate that local microinjection of a lipid/IL-22 cDNA vector complex is potentially a powerful strategy for treating colitis.

Although microinjection is effective, its clinical application has been hampered by the general requirement for surgery and specific injection skills. In contrast, oral delivery offers a more accessible treatment strategy that is conducive to patient compliance. From this aspect, developing an oral gene delivery method for IBD treatment appears to have more translational value than microinjection. There remains a need for improved compositions for delivery of active agents by oral administration for the treatment of inflammatory conditions.

Therefore, it is an object to provide lipid nanoparticles and pharmaceutical compositions thereof, for oral delivery of RNA-based therapeutics.

It is another object provide methods for treating inflammatory diseases and disorders using the lipid nanoparticles, and pharmaceutical compositions thereof.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### BRIEF SUMMARY OF THE INVENTION

Disclosed are compositions, systems, and methods involving lipid nanoparticle primarily composed of phosphatidic acid (PA), monogalactosyldiacylglycerol (MGDG), and digalactosyldiacylglycerol (DGDG). In particular, the PA, MGDG, and DGDG are present in the nanoparticles in useful ratios, preferably falling in a ratio of 3 to 7, 1 to 3, and 2 to 4, respectively. Further, it is useful for the PA, MGDG, and DGDG to make up 90% or more of the total lipid in the nanoparticles. The disclosed lipid nanoparticles are useful as drug delivery systems for delivery of a drug, such as oral deliver, intravascular delivery, or intramuscular delivery. The disclosed lipid nanoparticles can be used in methods involving administration or delivery of the nanoparticles to a subject. In some forms, the subject can be a disease or condition, such as inflammatory bowel disease, ulcerative colitis, Crohn's disease, cancer, colon cancer, or coronavirus infection.

Disclosed are lipid nanoparticles including phosphatidic acid (PA), monogalactosyldiacylglycerol (MGDG), and digalactosyldiacylglycerol (DGDG), where the PA, MGDG, and DGDG are present in the nanoparticle in a ratio of 3 to 7, 1 to 3, and 2 to 4, respectively, and where the PA, MGDG, and DGDG make up 90% or more of the total lipid in the nanoparticle. In some forms, the PA, MGDG, and DGDG are each of high purity before being mixed to form the nanoparticle. In some forms, no component of the nanoparticle is obtained from ginger. In some forms, the PA, MGDG, and DGDG are present in the nanoparticle in a ratio of 5:2:3.

In some forms, the PA, MGDG, and DGDG make up 95% or more of the total lipid in the nanoparticle. In some forms, the PA, MGDG, and DGDG make up 97% or more of the total lipid in the nanoparticle. In some forms, the PA, MGDG, and DGDG make up 99% or more of the total lipid in the nanoparticle. In some forms, the PA, MGDG, and DGDG are each of at least 97% purity before being mixed to form the nanoparticle. In some forms, the PA, MGDG, and DGDG are each of at least 98% purity before being mixed to form the nanoparticle. In some forms, the PA, MGDG, and DGDG are each of at least 99% purity before being mixed to form the nanoparticle.

In some forms, the lipid nanoparticle further include ionizable lipids, where the ionizable lipids make up 10% or less of the total lipid in the nanoparticle. In some forms, the ionizable lipids comprise cationic lipids. In some forms, the lipid nanoparticles further include one or more surface modifications.

In some forms, the lipid nanoparticles further include one or more payload components. In some forms, at least one of the payload components is a compound for delivery to intestine. In some forms, at least one of the payload components is a therapeutic agent for treatment of inflammatory bowel disease. In some forms, at least one of the payload components is a therapeutic agent for treatment of ulcerative colitis. In some forms, at least one of the payload components is a therapeutic agent for treatment of Crohn's disease. In some forms, at least one of the payload components is a therapeutic agent for treatment of cancer. In some forms, at least one of the payload components is a therapeutic agent for treatment of colon cancer. In some forms, at least one of the payload components is a therapeutic agent for treatment of coronavirus infection.

In some forms, at least one of the payload components is RNA. In some forms, at least one of the payload components is mRNA. In some forms, at least one of the payload components is an mRNA encoding IL-22. In some forms, at least one of the payload components is siRNA or a replicate for siRNA. In some forms, at least one of the payload components is DNA. In some forms, at least one of the payload components is pDNA. In some forms, at least one of the payload components is a protein.

Also disclosed are pharmaceutical compositions that include the disclosed lipid nanoparticles. In some forms, the pharmaceutical composition is formulated for oral administration. In some forms, the pharmaceutical composition is formulated for intravascular administration. In some forms, the pharmaceutical composition is formulated for intramuscular administration.

Also disclosed are methods involving administering the disclosed pharmaceutical compositions to a subject. In some forms, the composition is administered orally to the subject. In some forms, the composition is administered intravascularly to the subject. In some forms, the composition is administered intramuscularly to the subject.

In some forms, the subject is suffering a disease or condition. In some forms, the subject is suffering inflammatory bowel disease. In some forms, the subject is suffering ulcerative colitis. In some forms, the subject is suffering Crohn's disease. In some forms, the subject is suffering cancer. In some forms, the subject is suffering colon cancer. In some forms, the subject is suffering coronavirus infection.

In some forms, the pharmaceutical composition is for use in oral delivery of a drug. In some forms, the pharmaceutical composition is for use in intravascular delivery of a drug. In some forms, the pharmaceutical composition is for use in intramuscular delivery of a drug.

Also disclosed are drug delivery systems for delivery of a drug, where the drug delivery system includes the disclosed lipid nanoparticles. In some forms, the drug delivery system is formulated for oral delivery. In some forms, the drug delivery system is formulated for intravascular delivery. In some forms, the drug delivery system is formulated for intramuscular delivery.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings illustrate several embodiments of the disclosed method and compositions and together with the description, serve to explain the principles of the disclosed method and compositions.

FIG. 1. Simplified schematic of synthetic IL-22 mRNA generation via in vitro transcription (IVT). Plasmid DNA was designed to contain the IL-22 gene. IVT, 5′ capping, and polyA tail attachment are steps for improving mRNA stability. Multiple centrifugations generate purified synthetic IL-22 mRNA.

FIGS. 2A-2F. Characterization of nLNPs and stability test of IL-22/nLNPs. (A-B) Size and zeta potential of blank nLNPs and IL-22/nLNPs (n=3), (C) Representative thin layer chromatography (TLC) for nLNP compounds separation (from left to right D: DGDG, M: MGDG, P: PA, N: nLNPs, G: GDNPs), (D) Representative SEM images of blank nLNPs and IL-22 loaded nLNPs, the yellow color was added by Adobe Photoshop (version CS6) allows for easier lipid nanoparticles identification and differentiation, (E) Stability test of IL-22 mRNA exposed to RNase (with or without nLNPs protection) by 2% agarose gel electrophoresis, and (F) Size and zeta potential stability test of blank nLNP and IL-22/nLNP in PBS solution at room temperature.

FIG. 3. IL-22 mRNA is more efficient than IL-22 pDNA for expressing IL-22 protein in intestinal epithelial cells (Caco2-BBE). Significant increase of IL-22 by transfection of nLNPs encapsulated IL-22 mRNA compared to encapsulated pDNA or controls (*P<0.05, ****P<0.0001, n=6).

FIGS. 4A-4F. In vivo treatment of IL-22 pDNA and mRNA and protein expression level in three parts of the colon, including proximal, middle, and distal. (A-C) 3-day-treatment of IL-22 pDNA and euthanized 4 days later, (D-F) 3-day-treatment of IL-22 mRNA and euthanized 1 day later (*P<0.05, ns: nonsignificant, n=3).

FIGS. 5A-5F. Oral administration of IL-22/nLNPs accelerates healing in a mouse model of intestinal wound healing (A) Timeline of IL-22-mRNA treatment (7 days) on DSS-wounded mice (7 days), (B) Body weight change, (C) Colon length, (D) Fecal lipocalin-2 level, (E) IL-22 concentration, and (F) Colonic myeloperoxidase (MPO) level after 7 day-treatment (*P<0.05, ***P<0.001, ns: nonsignificant, n=5).

FIG. 6. Oral delivery of IL-22/nLNPs suppresses colonic expression of pro-inflammatory cytokines. Quantitative measurement of pro-inflammatory cytokines alteration (TNF-α, IL-6, and IL-1b) (*P<0.05, **P<0.01, ns: nonsignificant, n=5).

FIGS. 7A-7D. H&E-stained and Ki-67 (proliferation)-stained colon tissues after treatment of IL-22/nLNPs on DSS-wounded mice. (A) Top images (5×) and bottom images (20×) show magnification of stained colon tissues for each group (healthy, DSS+PBS, and DSS+IL-22/nLNPs), (B) Ki-67 staining image of colon tissues, (C) histology score of H&E staining, and (D) proliferation cell percentage of Ki-67-stained colon tissues (*P<0.05).

FIGS. 8A-8H. Oral administration of IL-22/nLNPs had no noticeable impact on the gut microbiota composition. (A) Observed taxa showing diversity calculated based on the operational taxonomic unit (OTU), (B) Principal coordinate analysis (PCoA) of the weighted UniFrac distance matrix of fecal microbiota from untreated, PBS, blank nLNPs, and IL-22/nLNPs group, (C) Donut graph of microbial composition at the phylum level, (D) Venn diagram analysis of common bacterial phylum of each group, (E) heatmap of relative abundance of microbial distinguishment at the phylum level, (F) relative abundance of the microbiome at the phylum level, (G) relative abundance in percentages of Bacteroidetes and firmicutes, and (H) Firmicute to Bacteroidetes ratio (*P<0.05, ns: nonsignificant, n=5).

FIGS. 9A-9F. Complete blood cell count (A) white blood cell (B) lymphocyte (C) neutrophil (D) mononuclear (E) red blood cell (F) platelet.

FIGS. 10A-10N. Blood biochemistry analysis (FIG. 10A) aminotransferase (FIG. 10B) albumin (FIG. 10C) alkaline phosphatase (FIG. 10D) amylase (FIG. 10E) bilirubin (FIG. 10F) nitrogen (FIG. 10G) calcium (FIG. 10H) phosphorus (FIG. 10I) creatine (FIG. 10J) glucose (FIG. 10K) sodium (FIG. 10L) potassium (FIG. 10M) total protein (FIG. 10N) globulin.

FIGS. 11A and 11B. Microbiome analysis: species (FIG. 11A) bar graph of relative abundance of microbiome at species level (FIG. 11B) heatmap of relative abundance of microbial distinguishment at species level.

FIGS. 12A and 12B. Microbiome analysis: family (FIG. 12A) bar graph of relative abundance of microbiome at family level (FIG. 12B) heatmap of relative abundance of microbial distinguishment at family level.

FIG. 13 shows representative chemical structures of lipids at a ratio of 5:3:2 (Pie chart) for phosphatidic acid (PA), digalactosyldiacylglycerol (DGDG), and monogalactosyldiacylglycerol (MGDG), respectively.

FIG. 14 is a bar graph showing loading efficiency of IL-22 mRNA to nLNPs (67-74%).

FIG. 15 is a bar graph showing in vitro transfection efficiency comparison between IL-22/nLNPs and IL-22/GDNPs (****p<0.0001, ns: nonsignificant, n=12).

FIG. 16 are bar graphs showing spleen weight (left) and spleen to body weight ratio (right) comparing DSS wounded and IL-22/nLNPs treated mice to other groups (healthy, PBS-treated, and blank nLNPs-treated). (*p<0.05, ns: nonsignificant, n=3).

FIGS. 17A and 17B show biodistribution of DiR-tagged IL-22/nLNPs in mouse organs after 7-day consecutive oral administration. FIG. 17A is a bar graph showing quantitative measurement of fluorescence detected in mouse organs (stomach, caecum, lung, brain, kidney, spleen, heart, liver, colon, and small intestine). FIG. 17B is a dot plot showing Serum IL-22 quantitation by ELISA test for samples from each group (healthy, PBS, Blank nLNPs, and IL-22/LNPs). (****p<0.0001, ns: nonsignificant, n=4 or 5 mice per group).

### DETAILED DESCRIPTION OF THE INVENTION

The disclosed method and compositions may be understood more readily by reference to the following detailed description of particular embodiments and the Example included therein and to the Figures and their previous and following description.

### I. Definitions

“Self-assembling,” “self-assembly,” and related terms, as used herein, refer to the use of an amphiphilic bottlebrush block copolymers, alone or in mixture with lipids and/or hydrophobic polymers, which orient in a mixture of aqueous and non-aqueous solvents to form lipid nanoparticles, wherein the hydrophilic ends orient with the other hydrophilic ends and the hydrophobic ends orient with the other hydrophobic ends.

“Pharmaceutically acceptable,” as used herein, refers to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration.

Use of the term “about” is intended to describe values either above or below the stated value, which the term “about” modifies, to be within a range of approximately +/−10%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise.

° C., where the range also discloses temperatures that can be selected independently from about 25, 26, 27, 28, 29, and 30° C., as well as any range between these numbers (for example, 26 to 28° C.), and any possible combination of ranges between these values.

Use of the term “about” is intended to describe values either above or below the stated value, which the term “about” modifies, to be within a range of approximately +/−10%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise.

“Analog” as relates to a given compound, refers to another compound that is structurally similar, functionally similar, or both, to the specified compound. Structural similarity can be determined using any criterion known in the art, such as the Tanimoto coefficient that provides a quantitative measure of similarity between two compounds based on their molecular descriptors. Preferably, the molecular descriptors are 2D properties such as fingerprints, topological indices, and maximum common substructures, or 3D properties such as overall shape, and molecular fields. Tanimoto coefficients range between zero and one, inclusive, for dissimilar and identical pairs of molecules, respectively. A compound can be considered an analog of a specified compound, if it has a Tanimoto coefficient with the specified compound between 0.5 and 1.0, inclusive, preferably between 0.7 and 1.0, inclusive, most preferably between 0.85 and 1.0, inclusive. A compound is functionally similar to a specified, if it induces the same pharmacological effect, physiological effect, or both, as the specified compound. “Analog” can also refer to a modification including, but not limited to, hydrolysis, reduction, or oxidation products, of the disclosed compounds. Hydrolysis, reduction, and oxidation reactions are known in the art.

Disclosed are compositions, systems, and methods involving lipid nanoparticle primarily composed of phosphatidic acid (PA), monogalactosyldiacylglycerol (MGDG), and digalactosyldiacylglycerol (DGDG). In particular, the PA, MGDG, and DGDG are present in the nanoparticles in useful ratios, preferably falling in a ratio of 3 to 7, 1 to 3, and 2 to 4, respectively. Further, it is useful for the PA, MGDG, and DGDG to make up 90% or more of the total lipid in the nanoparticles. The disclosed lipid nanoparticles are useful as drug delivery systems for delivery of a drug, such as oral deliver, intravascular delivery, or intramuscular delivery. The disclosed lipid nanoparticles can be used in methods involving administration or delivery of the nanoparticles to a subject. In some forms, the subject can be a disease or condition, such as inflammatory bowel disease, ulcerative colitis, Crohn's disease, cancer, colon cancer, or coronavirus infection.

### II. Compositions

Lipid nanoparticles containing phosphatidic acid (PA), monogalactosyldiacylglycerol (MGDG), and digalactosyldiacylglycerol (DGDG) have been developed. The lipid nanoparticles disclosed herein are suitable for encapsulating nucleic acids e.g., mRNA for oral delivery.

Pharmaceutical compositions containing the lipid nanoparticles are also disclosed. These pharmaceutical compositions are suitable for oral, intravascular, or intramuscular delivery to a subject in need thereof, for treating inflammatory diseases and disorders, or inflammation associated with a disease or disorder.

### A. Lipid Nanoparticles

Disclosed are lipid nanoparticles including phosphatidic acid (PA), monogalactosyldiacylglycerol (MGDG), and digalactosyldiacylglycerol (DGDG). Phosphatidic acid (PA), monogalactosyldiacylglycerol (MGDG), and digalactosyldiacylglycerol (DGDG) are represented below by Structures I, II, and III, respectively.

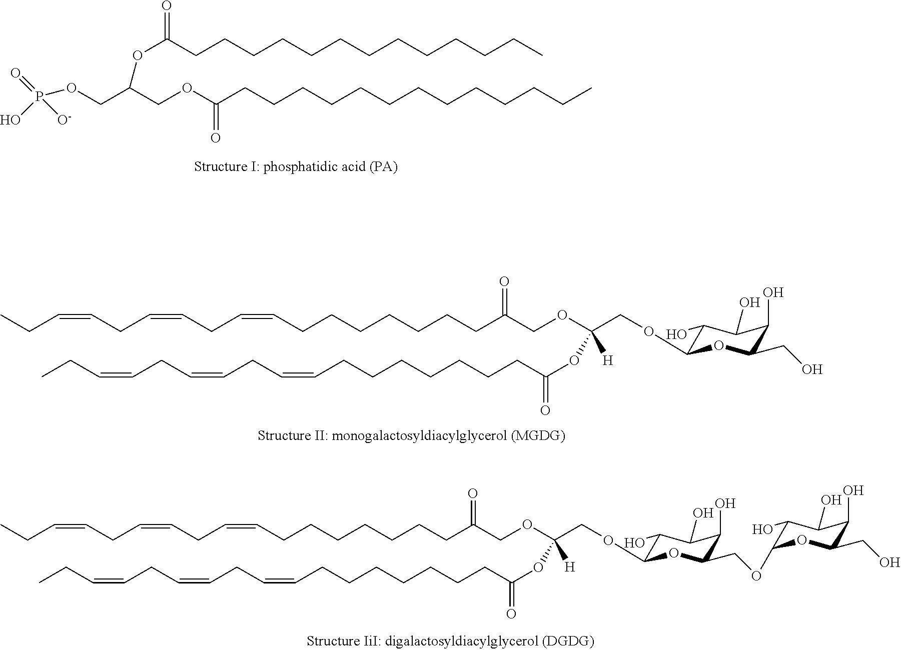

In some forms, the PA, MGDG, and DGDG are present in the nanoparticle in a ratio of 3 to 7, 1 to 3, and 2 to 4, respectively, and where the PA, MGDG, and DGDG make up 90% or more of the total lipid in the nanoparticle. In some forms, the PA, t0 MGDG, and DGDG are each of high purity before being mixed to form the nanoparticle. In some forms, no component of the nanoparticle is obtained from ginger. In some forms, the PA, MGDG, and DGDG are present in the nanoparticle in a ratio of 5:2:3.

In some forms, the PA, MGDG, and DGDG make up 95% or more of the total lipid in the nanoparticle. In some forms, the PA, MGDG, and DGDG make up 97% or more of the total lipid in the nanoparticle. In some forms, the PA, MGDG, and DGDG make up 99% or more of the total lipid in the nanoparticle. In some forms, the PA, MGDG, and DGDG are each of at least 97% purity before being mixed to form the nanoparticle. In some forms, the PA, MGDG, and DGDG are each of at least 98% purity before being mixed to form the nanoparticle. In some forms, the PA, MGDG, and DGDG are each of at least 99% purity before being mixed to form the nanoparticle.

In some forms, the lipid nanoparticle further includes ionizable lipids, helper lipids, or combinations thereof, to increase the LNPs' stability and fluidity. Preferably, the ionizable lipids make up 10% or less of the total lipid in the nanoparticle. In some forms, the ionizable lipids comprise cationic lipids. Exemplary helper lipid molecules include, but are not limited to, phospholipids, sphingolipids, saturated fatty acid, unsaturated fatty acids, glycerides, monoglycerides, diglycerides, triglycerides, hormones, steroids (e.g., cholesterol, bile acids), vitamins (e.g., vitamin E), or combinations thereof. In some forms, the lipid nanoparticles further include one or more surface modifications.

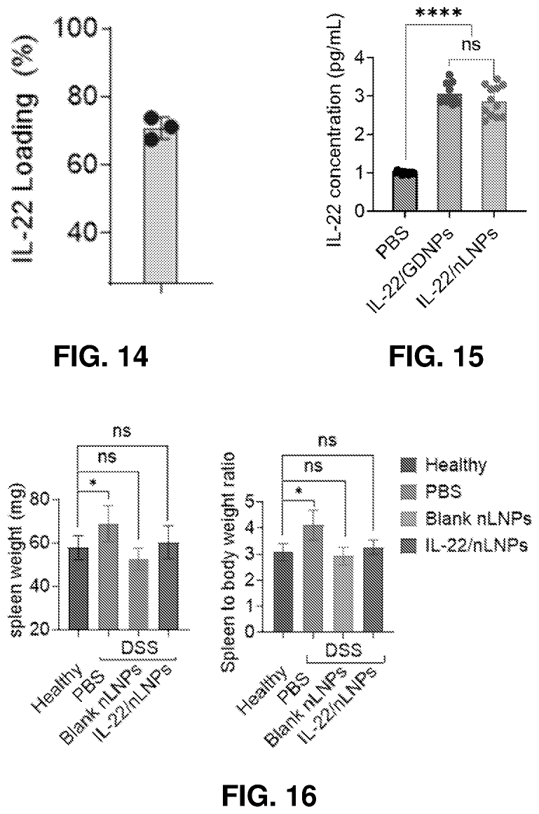

The lipid nanoparticles generally have a loading efficiency from about 10% to about 80% of the payload component. “Loading efficiency” or “drug loading efficiency”, as used herein, refers to the ratio of the amount of drug in the nanoparticle to the total amount of drug applied in formulation of the nanoparticles. For example, as described in the non-limiting Examples, the lipid nanoparticles have a loading efficiency of IL-22 mRNA to LNPs of about 67% to about 74% (see FIG. 14). Thus in some forms, the lipid nanoparticles have a loading efficiency of about 10%, about 20%, about 30%, about 40%, about 50%, about 60%, about 70% or about 80% of the payload component.

### 1. Lipid Nanoparticle Size

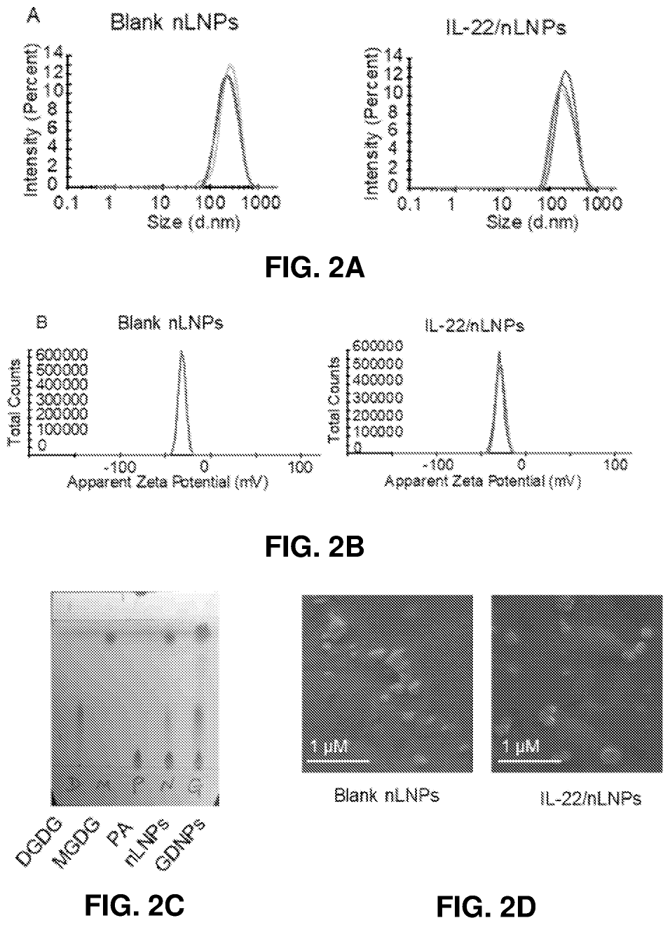

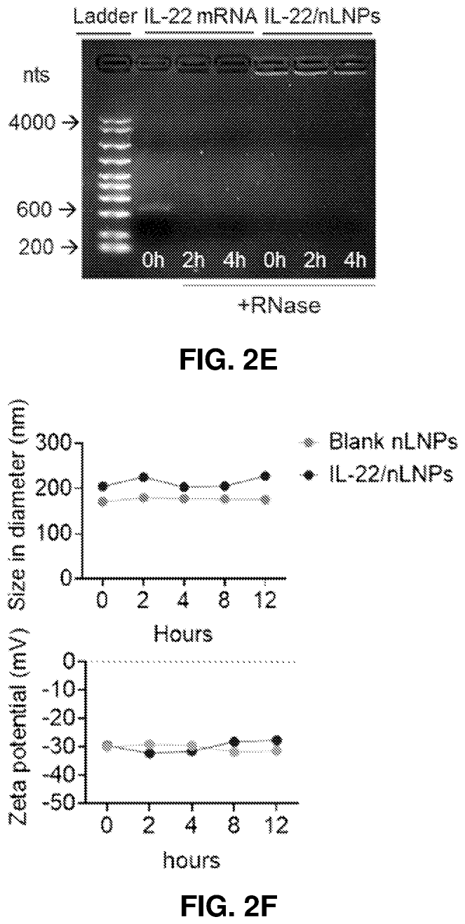

The LNPs typically have a particle diameter between 100 nm and less than 1,000 nm, as measured using dynamic light scattering, as demonstrated in the non-limiting examples and FIGS. 2A and 2F. The diameter can be the mean hydrodynamic diameter of the LNPs. The term “hydrodynamic diameter” refers to the diameter of a lipid nanoparticle.

In some forms, the LNPs have a mean hydrodynamic diameter between about 100 nm and about 1000 nm, between about 100 nm and about 500 nm, or between about 100 nm and about 300 nm, as measured using dynamic light scattering. In some forms, the LNPs have a mean hydrodynamic diameter of about 100 nm, about 200 nm, about 250 nm, about 300 nm, about 350 nm, about 400 nm, about 450 nm, about 500 nm, about 600 nm, about 700 nm, about 800 nm, about 900 nm, or about 1000 nm as measured using dynamic light scattering. In preferred forms, the LNPs have a mean hydrodynamic diameter of about 100 nm to about 300 nm, more preferably about 150 nm to about 250 nm, most preferably, about 200 nm as measured using dynamic light scattering. Based on whether delivery is oral or systemic, such as to the systemic circulation, such as to the GI tract or other mucosal surfaces in a mammal, the nanoparticles can have different size sub-ranges within this range. As demonstrated in the non-limiting Examples, the size of both the empty LNPs and LNPs containing IL-22 were maintained in PBS solution for more than 5 hours (FIG. 2F).

### 2. Zeta Potential

The surface of the LNPs is/are neutral or negatively or positively charged or contain(s) one or more moieties (i.e., one or more functional groups) that impart a negative or positive charge to the LNPs in water or Phosphate-Buffer Saline (PBS). Optionally, these moieties are present on the surface of the LNPs, such that the surface of LNPs itself display negative or positive zeta potentials in DI water. “Zeta potential” as used herein refers to a measure of the effective electric charge on the nanoparticle's surface, quantifying the charges. When a nanoparticle has a net surface charge, the charge is screened by the concentration of ions of opposite charge near the nanoparticle surface. In some forms, the lipid nanoparticles have a zeta potential in a range from −10 mV and −80 mV, such as from −20 mV to −70 mV, in DI water at room temperature. In preferred forms, the lipid nanoparticles have a zeta potential in a range from about −25 mV to about −40 mV, more preferably about −29±0.5 mV. As demonstrated in the non-limiting Examples, the zeta potential for both the empty LNPs and LNPs containing IL-22 were maintained in PBS solution for more than 5 hours (FIG. 2F).

### 3. Payload Components

The lipid nanoparticles further include one or more payload components, which refer to therapeutic agents, drugs, genetic materials, or other biologically active substances that can be incorporated into the lipid nanoparticle for delivery to a specific target site e.g., the intestine. In some forms, the payload component is encapsulated within the lipid nanoparticle, a process that involves enclosing the payload component within the lipid bilayer or core of the nanoparticle structure.

During the preparation of the lipid nanoparticles, the payload component(s) are integrated into the lipid matrix through various encapsulation techniques, known in the art. These techniques may involve the use of solvent-based methods, such as solvent evaporation or emulsification, or solvent-free methods like thin-film hydration or high-pressure homogenization. Regardless of the method employed, the process ensures that the payload component(s) become entrapped or embedded within the lipid bilayers or the internal aqueous core of the lipid nanoparticle.

In some forms, at least one of the payload components is a compound for delivery to intestine. In some forms, at least one of the payload components is a therapeutic agent for treatment of inflammatory bowel disease. In some forms, at least one of the payload components is a therapeutic agent for treatment of ulcerative colitis. In some forms, at least one of the payload components is a therapeutic agent for treatment of Crohn's disease. In some forms, at least one of the payload components is a therapeutic agent for treatment of cancer. In some forms, at least one of the payload components is a therapeutic agent for treatment of colon cancer. In some forms, at least one of the payload components is a therapeutic agent for treatment of coronavirus infection.

In some forms, at least one of the payload components is RNA. In some forms, at least one of the payload components is mRNA. In some forms, at least one of the payload components is an mRNA encoding IL-22. The DNA sequence for mouse IL-22 is represented by SEQ ID NO:1. The DNA sequence for human IL-22 is represented by SEQ ID NO:18. In some forms, the mRNA encoding IL-22 is transcribed from SEQ ID NO:1 or SEQ ID NO: 18. In some forms, the mRNA is transcribed from a DNA sequence having sequence identity of about 75%, about 80%, about 90%, about 95%, or about 99% of SEQ ID NO:1 or SEQ ID NO:18.

IL-22 DNA Sequence (Organism: Mus musculus, Gene ID: 50929)
(SEQ ID NO: 1)
CATGGCTTTCTTTGAAAACTGTGTTTAGAAGATTTCTGGGATTTGTGTGCAAAAGCACCTTGTTGGCCCTC
ACCGTGACGTTTTAGGGAAGACTTCCCATCTCTCAAGGTGGGAAGGCTTGGAGGTGGTGTCTTGTGGCCTC
CTATGGTGGTTAGGTACTTCTCAGAAGACAGGACTGGAAATTAGATAATGTCTGATGTCATATCATTCACA
ATACCAAAAAAACCCTGGTGTCCCGATGGCTATAAAAGCAGCAACTTCTGCCTCTCCCATCACAAGCAGAG
ACACCTAAACAGGCTCTCCTCTCAGTTATCAACTGTTGACACTTGTGCGATCTCTGATGGCTGTCCTGCAG
AAATCTATGAGTTTTTCCCTTATGGGGACTTTGGCCGCCAGCTGCCTGCTTCTCATTGCCCTGTGGGCCCA
GGAGGCAAATGCGCTGCCCGTCAACACCCGGTGCAAGCTTGAGGTGTCCAACTTCCAGCAGCCATACATCG
TCAACCGCACCTTTATGCTGGCCAAGGAGGCCAGCCTTGCAGATAACAACACAGATGTCCGGCTCATCGGG
GAGAAACTGTTCCGAGGAGTCAGTGCTAAGGATCAGTGCTACCTGATGAAGCAGGTGCTCAACTTCACCCT
GGAAGACGTTCTGCTCCCCCAGTCAGACAGGTTCCAGCCCTACATGCAGGAGGTGGTGCCTTTCCTGACCA
AACTCAGCAATCAGCTCAGCTCCTGTCACATCAGCGGTGACGACCAGAACATCCAGAAGAATGTCAGAAGG
CTGAAGGAGACAGTGAAAAAGCTTGGAGAGAGTGGAGAGATCAAGGCGATTGGGGAACTGGACCTGCTGTT
TATGTCTCTGAGAAATGCTTGCGTCTGAGCGAGAAGAAGCTAGAAAACGAAGAACTGCTCCTTCCTGCCTT
CTAAAAA.
IL-22 DNA Sequence (Organism: Homo sapiens, NCBI Gene ID: 50616):
(SEQ ID NO: 18)
ACAAGCAGAATCTTCAGAACAGGTAAGCGTTTCGGCAAACTTGGTACAATTGGTTAGTTTGATGAAATACT
TCTTGACTAATTTTGTTCCTTCACGTTGTCTTCGACCAGGTTCTCCTTCCCCAGTCACCAGTTGCTCGAGT
TAGAATTGTCTGCAATGGCCGCCCTGCAGAAATCTGTGAGCTCTTTCCTTATGGGGACCCTGGCCACCAGC
TGCCTCCTTCTCTTGGCCCTCTTGGTACAGGGAGGAGCAGCTGCGCCCATCAGCTCCCACTGCAGGCTTGA
CAAGTCCAACTTCCAGCAGCCCTATATCACCAACCGCACCTTCATGCTGGCTAAGGAGGTATACATCTCAA
TCCTGCTCTTTCTCGTTGGATCTACTTGGAATCCAAATAGTTCTTAAACTTTTCTTCAGAGCATCTCTAAG
AGCTTTAGGAACCCACTGTTTATCCCTGAGGGTAGATAAATTTTCTGTTTTTTCAGAGACTCTTTGGGAAT
CTGGCTTTTTTTTTTTTCTTGAACTTCTTCCTTCCATTTTGGCCTTTATGATACATATGATGAATTTTTCC
CAAAGAGCGGCCATTCAGTAATCCATCTGATGATTTTTTTTTTCCTTTATGCCTCTGTGCATTGTTCTAAA
CTCATGCACACATCTGAATTCTGCTTTTAGTCTTTATGATGTTGCTCTGGGGAGACGGGATGGGGCACATG
TCTATGTATAAATTTTTTTTCTATTTGCTCAATGTCCAGACCCTTAGTCTTTTCTTCTCTTCCAGGCTAGC
TTGGCTGATAACAACACAGACGTTCGTCTCATTGGGGAGAAACTGTTCCACGGAGTCAGTGTAAGCTACAG
TTGTGACGAACAGGGCCGTGTGCCGTCCATGGGTACTTGGGGTGGTGGTGATGATGGTTTAGGTCTTATCC
CTTATGACCCTTTCTGTTTCCCTTCCACCTGCAGATGAGTGAGCGCTGCTATCTGATGAAGCAGGTGCTGA
ACTTCACCCTTGAAGAAGTGCTGTTCCCTCAATCTGATAGGTTCCAGCCTTATATGCAGGAGGTGGTGCCC
TTCCTGGCCAGGCTCAGCAACAGGCTAAGCACATGTGTAAGTTCAGCTCTCAGCCTATGCCCACCTACCCC
TCCTTCCCTCCTTCCACAGAGACCCCCTTACCCCAACTCTCTCTCCTTCCCCCTACCCCTAAGCTAGCAGG
AAGAAGTGTCTTGGCAGCAGTGTTATCAGGAGTCATTTGGGATCATAGAGTATTTGCTTTTGCTTTGACTG
AGTCACATCTTGAGTTTATAGTGGTGAATGGGGTCTGGAACTTAAGTGTACAGAAGCCGCATTGGTTTGTC
TTCGGAAAAAAGGCAACTCAGGTTGCGTAAGATGAGAAAGGTGTTGGGAAAAACATCTAGATGTGGAAATG
GATCCATTGAGTCTAAGTTGTTGAGGGGAGGGGATGGCATGGAGAGAAATTAGAAGAGAAAGTGGGAAATG
GGAAGGCTTAAAGTCGGTGGTGGGTCGGCAGACTGTTGCCCTGTTGATGTCATGGGAAGCCACAAAATCGG
AGGCGTGTGAACTTGATGCCGCTGAACATTTGAAACTATGAAAAAAAGTTTGAGTGGAGTGGGCCCAGTAA
AAGGCCCTAGGACTTACTGAAGAGGGCTTAATTTTCACATGAGATGTTTTATGTACATTTCTTGTTCTAAG
CATGCAATTTTCTGGAGATACGATTGAGGTTTTATTCCTTACAGAATTTGCATAAACTACTCCGCTCTTTC
CACAAATGCAAACCTCAGTAGGATTTCCCAAAGATGAAGAGAGGTCTCTTGTAAGGGAAGTGACTGGATTC
TGGCGTCCAAGGGAATTCAAGAGCTCAGGAAATCTAGGTCACTGTTGAAATCTAGGTCATTGTGGGCAAAA
TTACTAAGAGCTTTAATTCCAGGTGAATTGTACTGTACCTCCATGGGTGTGGAGGTTCATAAAGTTTCAGC
ACAACATTAAGATAGTTATGCTTGTTATTGTTTTATAGCATATTGAAGGTGATGACCTGCATATCCAGAGG
AATGTGCAAAAGCTGAAGGACACAGTGAAAAAGGTAGGACTGATAACTGTCAATGCTAAGTCATGCAATAG
GAGAGACAAATGTTGTTTTTCTTTCCTTTCTTTCTTCCCATCACTTTGTGATTTTTCACTTGATTCTCCTA
CCACCAGGGCGATTACTTTGGTGTCTGTGTATGTAGATATATCTATATATCTAGATGTCAGTTTCCAAATC
TTGCAAATTGTAGAATTCTAGAACTGGTTGGGATCTTAGCTTGTCTAGTCACATAACCTCAGATTCTGGGG
ATGGTCAGTGGCAGAGATAGGGCTAGAATGCAGGTCTCCTGAATCCCAAGCCAGCACTTTTCCCGGTGGTG
ATACAGATTAGTTTTGGTACCATTAATTCTTAGGGAAATTTCAGATTCCTATTGACTCATGTAATCTGAAG
AAGTACTTGTTTAAAAACAGAAAAATGCCTATGGGCAAATTTATTTGAAGTCATTTTTGAAGTCATTAATG
CATTGCTTTGAAACTTGGAAGAATAAACTCAGAACAATGAGAAAAGAGCTGGACTTGCATATAGGGCTAAT
TTCTGGAGTAATAAACACTTATTTTGAATTATCATAATATCTATCAGATATTGATTATAGTTTAAAAGCAA
GAGCAGACAACCCCGATCTCTTTTATACAGGTTCAAATAGAGTAAAAATATTAGTAAGAGATTTATTAAAG
TTAAATGGAAGTCTGAATTGGTAAGCTTTTTTTTCTTCCTCTCTCCCATCAAGACCTTCCATTCTAGTTTC
TTCCTTCACTCCCTCAACAAATCCCTAGGGAGCATTTATCCATGGTGGGCTGGTGTACATTTCTATAGTGA
ATGATACCATCATGTGGCCTATTTGGTGAAAAGAACAACAATGGAAGGCTTAGACTAACAATAGTGACTCA
CCCCAAAACCGGAGGAATGATTAGGAGCAGTGAAAGTGACGCTCTTGCAAGCAGGTACAACTAAATACTCA
GAAACATGAAGGCTCCAGTTGATGGAATTTTCAGTAACAAGCTTAACCTTAATTCCCCCTTTTTCCCTCTT
GACTTTTTAAAAAAGCGTTTCTTCCTGAGCATCATTTAATGAGTGTGACTGTTTCTTCCTTTGATAATTGA
AGGCTTTGTAGTTTTAAATTGTGAAGCCCAGTTCTCTTGTTATAGAACTATTATCTAGACATGGAGGGCTG
AATGTTAGCATGCCACAGACAAGGCATGCTTTACACATCTTGCTTAAAAAATTACTGATTTCATCTTGCTT
GTTGTCTTTAGAAAAGTGAAGTGTGAGAGAGGAGAATCTCATGGTGATCTGTGTGATTTTCAAGACCTTTA
ATCCATTTTGAAAGAATCAATTTCATATTTGCAATGGGTTGCCATGTGGAAGAGTGATTATGCTTTTTTGC
TGGTAGCTTCAGAAAGCACAGGAGGGAGAGCAATGTTGTTCAGAGAAAGATCAACAGGAGGAGAAACTGTC
AGAGCTGTCTGAAATAGGGTGGTTTTGGGAGGCATTAATTCCCTCTCGTTGGGGGTAAAAGCAGAACGCAG
GTTGGTAGTAAAATGCATGACAGACAGTAGGGGACGATAAACTTTAAAATTCTTTATAGTCTTGGAGTCTT
TGAGATAGAAAAGAATATCTTTTTGGCCTTATGTCAAAAGAAGTATGGAAAGGTGAAAGGGCGGAAGAAAG
CAGGAAAAGGAAGAACCATGTATTATACAGAGGACAATGGTGACAAGGTTTTTCTTGAAATAATGCAAATA
TGATAGATTAGAGGAATTTCAGTAGGGAATGCTTTTCACTTGAATTTGGGTTTCCTCTTCGATTAAGTTTG
GGATCCTCATCTGCATTTGACTTGGAGAGAGAAAGAATGAATGTTAGGACCTATATCTGGTTTTCTATTAA
CTAAAGCAAGTGGAAAAGACTTATTTGGTATTTTTCCCACAAAAGTGAAAACTTTTCTTTTACTGTTTGTC
AAAAAGGTGGAAATAGAAAAAGCCTTAATGTATTGGTGAATACATGGTTCAAAGTCATTTGAGTAGAGATG
TTTTAAATCAGGAGTGTCCAATCATTTGGCTTCCCTGGACCACCTTGAAAGAATTGTCTTGGTACACACAT
AAAATACAAGAACAATAGCTGATGAGCTAAAAAAGTCCATGCATAAATCTCATACTGTTTTAAGAAAGTTT
ATGAATTTCTGTTAGGGTGCATTCAAAGCTGTCCTGGGCCATGTGCGGCCTGTGGGCTGCAGGTTGGACAA
GCTCCTTATAAGTAATCTGTCATAGATAGTTTTGGAGCTGCAAAACAGGCCAAGGCATAATGGGTGGCACT
CGGGATCCCCCAGATCCCAGCCTCACTTCAGTCTCCTTGCTCTGGTTAAGAAGGGGTGGTCAACTCTCTGC
CCAGCTTTTAAACAGCTTCATTAGTGTGAGGTGCACCTGAAATTGATGCCTGCTGGTGGCCTCTCAGTCCA
GAGAGCCGTCATTTTAAGCTCTTTGGCAAATCATACAATACTAAAGGGATATTACTATGAATGTTTTACAA
ATGCTTAAAACTCGGTTTCTGTCTCCATCAACCTAATCTTGCAATTTCTAATTTGTTCACTTTAGAAAACA
TGGCATAAATGCTCAAATACTTTTGCATTCTTATTTTCACAGCTTGGAGAGAGTGGAGAGATCAAAGCAAT
TGGAGAACTGGATTTGCTGTTTATGTCTCTGAGAAATGCCTGCATTTGACCAGAGCAAAGCTGAAAAATGA
ATAACTAACCCCCTTTCCCTGCTAGAAATAACAATTAGATGCCCCAAAGCGATTTTTTTTAACCAAAAGGA
AGATGGGAAGCCAAACTCCATCATGATGGGTGGATTCCAAATGAACCCCTGCGTTAGTTACAAAGGAAACC
AATGCCACTTTTGTTTATAAGACCAGAAGGTAGACTTTCTAAGCATAGATATTTATTGATAACATTTCATT
GTAACTGGTGTTCTATACACAGAAAACAATTTATTTTTTAAATAATTGTCTTTTTCCATAAAAAAGATTAC
TTTCCATTCCTTTAGGGGAAAAAACCCCTAAATAGCTTCATGTTTCCATAATCAGTACTTTATATTTATAA
ATGTATTTATTATTATTATAAGACTGCATTTTATTTATATCATTTTATTAATATGGATTTATTTATAGAAA
CATCATTCGATATTGCTACTTGAGTGTAAGGCTAATATTGATATTTATGACAATAATTATAGAGCTATAAC
ATGTTTATTTGACCTCAATAAACACTTGGATATCCTAA

In some forms, the mRNA encodes a mouse IL-22 protein or a human IL-22 protein. The amino acid sequence for mouse IL-22 is represented by SEQ ID NO: 17. The amino acid sequence for human IL-22 is represented by SEQ ID NO:19. In some forms, the mRNA encodes an IL-22 protein having sequence identity of about 75%, about 80%, about 90%, about 95%, or about 99% of SEQ ID NO: 17 or SEQ ID NO:19.

IL-22 Amino Acid Sequence (Organism: Mus musculus, UniProt ID: Q9JJY9):
(SEQ ID NO: 17)
MAVLQKSMSFSLMGTLAASCLLLIALWAQEANALPVNTRCKLEVSNFQQPYIVNRTFMLAKEASLADNNTD
VRLIGEKLFRGVSAKDQCYLMKQVLNFTLEDVLLPQSDRFQPYMQEVVPFLTKLSNQLSSCHISGDDQNIQ
KNVRRLKETVKKLGESGEIKAIGELDLLFMSLRNACV
IL-22 Amino Acid Sequence (Organism: Homo sapiens, UniProt ID: Q9GZX6):
(SEQ ID NO: 19)
MAALQKSVSSFLMGTLATSCLLLLALLVQGGAAAPISSHCRLDKSNFQQPYITNRTFMLAKEASLADNNTD
VRLIGEKLFHGVSMSERCYLMKQVLNFTLEEVLFPQSDRFQPYMQEVVPFLARLSNRLSTCHIEGDDLHIQ
RNVQKLKDTVKKLGESGEIKAIGELDLLFMSLRNACI

In some forms, at least one of the payload components is siRNA or a replicate for t0 siRNA. In some forms, at least one of the payload components is DNA. In some forms, at least one of the payload components is pDNA. In some forms, at least one of the payload components is a protein.

### B. Pharmaceutical Compositions

Also disclosed are pharmaceutical compositions that include the disclosed lipid nanoparticles. In some forms, the pharmaceutical composition is formulated for oral administration. In some forms, the pharmaceutical composition is formulated for intravascular administration. In some forms, the pharmaceutical composition is formulated for intramuscular administration.

Typically, the amount of the lipid nanoparticles present in the pharmaceutical composition is present in an amount effective to treat an inflammatory disease or disorder in a subject e.g., inflammatory bowel disease, ulcerative colitis, or Crohn's disease. In some forms, the pharmaceutical composition is effective to treat inflammation associated with a disease or disorder in a subject e.g., cancer such as colon cancer. In some forms, the pharmaceutical composition is effective to treat inflammation associated with a viral infection in a subject e.g., a coronavirus infection.

The pharmaceutical compositions may also include one more pharmaceutically acceptable carrier and/or one or more pharmaceutically acceptable excipients. For example, the pharmaceutical composition may be in the form of a liquid, such as a solution or a suspension, and contain the disclosed lipid nanoparticle in an aqueous medium and, optionally, one or more suitable excipients for the liquid formulation. For example, the pharmaceutical composition may be in a solid form, such as a tablet or powders, and contain the disclosed lipid nanoparticles and one or more suitable excipients for the solid formulation.

Optionally, the pharmaceutical composition is in a liquid form, and contains an effective amount of the disclosed lipid nanoparticles in an aqueous medium and, optionally, one or more suitable excipients for the liquid formulation. Optionally, the pharmaceutical composition is in a solid form, and contains an effective amount of the disclosed lipid nanoparticles and one or more suitable excipients for a solid formulation. Optionally, the pharmaceutical composition may be in the form of a liquid, such as a solution or a suspension, and contain an effective amount of the lipid nanoparticles in an aqueous medium and, optionally, one or more suitable excipients for the liquid formulation. Optionally, the pharmaceutical composition is in a solid form, and contains an effective amount of the lipid nanoparticles and one or more suitable excipients for a solid formulation.

### 1. Carriers and Excipients

The pharmaceutical composition can contain one or more pharmaceutically acceptable carriers and/or one or more pharmaceutically acceptable excipients. Suitable pharmaceutically acceptable carriers and excipients are generally recognized as safe (GRAS), and may be administered to an individual without causing undesirable biological side effects or unwanted interactions.

Representative carriers and excipients that can be used in the pharmaceutical composition include solvents (including buffers), diluents, pH modifying agents, preservatives, antioxidants, suspending agents, wetting agents, viscosity modifiers, tonicity agents, and stabilizing agents, and a combination thereof.

In some forms, the compounds can be dissolved or suspended in a suitable carrier to form a liquid pharmaceutical composition, such as sterile saline, phosphate buffered saline (PBS), balanced salt solution (BSS), viscous gel, or other pharmaceutically acceptable carriers for administration. The pharmaceutical composition may also be a sterile solution, suspension, or emulsion in a nontoxic, parenterally acceptable diluent or solvent.

Excipients can be added to a liquid or solid pharmaceutical composition to assist in sterility, stability (e.g., shelf-life), integration, and to adjust and/or maintain pH or isotonicity of the compounds in the pharmaceutical composition, such as diluents, pH modifying agents, preservatives, antioxidants, suspending agents, wetting agents, viscosity modifiers, tonicity agents, and stabilizing agents, and a combination thereof.

### 2. Form

The pharmaceutical composition containing the lipid nanoparticles, can be in a liquid form or a solid form, as a liquid formulation or a solid formulation for oral administration or parenteral administration (e.g., intramuscular administration, intravenous administration, and intraperitoneal administration) to a subject.

a. Oral Formulations

In some forms, the pharmaceutical composition containing the lipid nanoparticle, can be in a form suitable for oral administration to a subject, such as a mammal (i.e., an oral formulation). Oral administration may involve swallowing, so that the lipid nanoparticles (s) enter the gastrointestinal tract, or buccal or sublingual administration may be employed by which the compound(s) enter(s) the blood stream directly from the mouth.

Formulations suitable for oral administration include solid formulations such as tablets, capsules containing particulates, liquids, powders, lozenges (including liquid-filled lozenges), chews, multi- and nano-particulates, gels, solid solutions, liposomes, films, ovules, sprays, and liquid formulations.

Liquid formulations for oral administration include suspensions, solutions, syrups, and elixirs. Such oral formulations may be employed as fillers in soft or hard capsules and can contain one or more suitable carriers and/or excipients, for example, water, ethanol, polyethylene glycol, propylene glycol, chitosan polymers and chitosan derivatives (e.g., N-trimethylene chloride chitosan, chitosan esters, chitosan modified with hydrophilic groups, such as amino groups, carboxyl groups, sulfate groups, etc.), methylcellulose, a suitable oil, one or more emulsifying agents, and/or suspending agents. Liquid formulations for oral administration may also be prepared by the reconstitution of a solid, for example, from a sachet.

Optionally, an effective amount of the lipid nanoparticles is included in a fast-dissolving and/or fast-disintegrating dosage form.

For tablet or capsule dosage forms, in addition to the lipid nanoparticles described herein, tablets generally contain disintegrants, binders, diluents, surface active agents, lubricants, glidants, antioxidants, colorants, flavoring agents, preservatives, or taste masking agents, or a combination thereof.

Examples of suitable disintegrants for forming a tablet or capsule dosage form include, but are not limited to, sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant can have a concentration in a range from about 1 wt % to about 25 wt %, from about 5 wt % to about 20 wt % of the tablet or capsule dosage form containing the lipid nanoparticles.

Binders are generally used to impart cohesive qualities to a tablet formulation. Suitable binders for forming a tablet or capsule formulation containing an effective amount of the lipid nanoparticles, include, but are not limited to, microcrystalline cellulose, gelatin, sugars, polyethylene glycol, natural and synthetic gums, polyvinylpyrrolidone, pregelatinized starch, chitosan polymers and chitosan derivatives (e.g. N-trimethylene chloride chitosan, chitosan esters, chitosan modified with hydrophilic groups, such as amino groups, carboxyl groups, sulfate groups, etc.), hydroxypropyl cellulose, and hydroxypropyl methylcellulose.

Suitable diluents for forming a tablet or capsule formulation include, but are not limited to, lactose (as, for example, the monohydrate, spray-dried monohydrate or anhydrous form), chitosan polymers and chitosan derivatives (e.g. N-trimethylene chloride chitosan, chitosan esters, chitosan modified with hydrophilic groups, such as amino groups, carboxyl groups, sulfate groups, etc.), N-sulfonated derivatives of chitosan, quaternarized derivatives of chitosan, carbosyalkylated chitosan, microcrystalline chitosan, mannitol, xylitol, dextrose, sucrose, sorbitol, microcrystalline cellulose, starch and dibasic calcium phosphate dihydrate.

Tablet or capsule formulations containing an effective amount of the lipid nanoparticles, may also contain surface active agents, such as sodium lauryl sulfate and polysorbate 80, and glidants such as silicon dioxide and talc. When present, surface active agents can have a concentration in a range from about 0.2 wt % to 5 wt % of the tablet or capsule formulation.

Tablet or capsule formulations containing an effective amount of the lipid nanoparticles, also can contain lubricants, such as magnesium stearate, calcium stearate, zinc stearate, sodium stearyl fumarate, and mixtures of magnesium stearate with sodium lauryl sulphate. Lubricants can have a concentration in a range from about 0.25 wt % to 10 wt %, from about 0.5 wt % to about 3 wt % of the tablet or capsule formulation.

Other possible excipients included in a tablet or capsule formulation containing an effective amount of the lipid nanoparticles, include glidants (e.g., Talc or colloidal anhydrous silica at about 0.1 wt % to about 3 wt % of the tablet or capsule formulation), antioxidants, colorants, flavoring agents, preservatives, and taste-masking agents. When present, glidants can have a concentration in a range from about 0.2 wt % to 1 wt % of the tablet or capsule formulation.

An exemplary tablet formulation contains up to about 80 wt % of the compound(s) described herein, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet or capsule blends, including an effective amount of the lipid nanoparticles, and one or more suitable excipients, may be compressed directly or by roller to form tablets. Tablet or capsule blends or portions of the blends may alternatively be wet-, dry-, or melt-granulated, melt congealed, or extruded before tableting. The final tablet or capsule formulation may contain one or more layers and may be coated or uncoated; it may even be encapsulated in a particle, such as a polymeric particle or a liposomal particle.

Solid formulations containing an effective amount of the lipid nanoparticles, for oral administration may be formulated to be immediate and/or modified release. Modified release formulations include delayed, sustained, pulsed, controlled, targeted, and programmed release formulations.

b. Parenteral Formulations

In some forms, the pharmaceutical composition containing an effective amount of the lipid nanoparticles, can be in a form suitable for administration directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intramuscular, and subcutaneous delivery. Suitable means for parenteral administration include needle (including microneedle) injectors, needle-free injectors, and infusion techniques.

For example, the pharmaceutical composition containing an effective amount of the lipid nanoparticles, is in a form suitable for intramuscular administration, intravenous administration, intraperitoneal administration, or subcutaneous administration, or a combination thereof. In preferred forms, the pharmaceutical composition containing an effective amount of the lipid nanoparticles is in a form suitable for intravascular delivery, or intramuscular delivery of a drug.

Parenteral formulations containing an effective amount of the lipid nanoparticles, described herein are typically aqueous solutions which can contain excipients such as salts, carbohydrates and buffering agents (e.g., from about pH 6.5 to about pH 8.0, from about pH 6.5 to about pH 7.4, from about pH 6.5 to about pH 7.0, from about pH 7.0 to pH 8.0, or from about pH 7.0 to about pH 7.4), but, for some applications, they may be more suitably formulated as a sterile aqueous solution or as a dried form to be used in conjunction with a suitable vehicle such as sterile, pyrogen-free water.

The liquid formulations containing an effective amount of the lipid nanoparticles, for parenteral administration may be a solution, a suspension, or an emulsion.

The liquid pharmaceutically acceptable carrier forming the parenteral formulation containing an effective amount of the lipid nanoparticles, can include one or more physiologically compatible buffers, such as a phosphate buffer. One skilled in the art can readily determine a suitable saline content and pH for an aqueous carrier for administration (e.g., from about pH 6.5 to about pH 8.0, from about pH 6.5 to about pH 7.4, from about pH 6.5 to about pH 7.0, from about pH 7.0 to pH 8.0, or from about pH 7.0 to about pH 7.4).

Liquid formulations containing an effective amount of the lipid nanoparticles, for parenteral administration may include one or more suspending agents, such as cellulose derivatives, sodium alginate, polyvinylpyrrolidone, gum tragacanth, or lecithin. The liquid formulations may also include one or more preservatives, such as ethyl or n-propyl p-hydroxybenzoate.

In some forms, the liquid formulation containing an effective amount of the lipid nanoparticles, contains one or more solvents that are low toxicity organic (i.e., nonaqueous) class 3 residual solvents, such as ethanol, acetone, ethyl acetate, tetrahydrofuran, ethyl ether, and propanol, and a combination thereof. Any such solvents included in the liquid formulation should not detrimentally react with the lipid nanoparticles, and any additional active agents when present in the liquid formulation. Solvents such as freon, alcohol, glycol, polyglycol, or fatty acid, can also be included in the liquid formulation containing the lipid nanoparticles, as desired to increase the volatility of the solution or suspension.

Liquid formulations containing the lipid nanoparticles, for parenteral administration may also contain minor amounts of polymers, surfactants, or other pharmaceutically acceptable excipients known to those in the art. In this context, “minor amounts” means an amount that is sufficiently small to avoid adversely affecting uptake of any of the lipid nanoparticles, by the targeted cells, such as pituitary gonadotrophs.

The preparation of parenteral formulations containing the lipid nanoparticles, is typically under sterile conditions, for example, by lyophilization, which can be accomplished using standard pharmaceutical techniques known to those skilled in the art.

Formulations for parenteral administration containing the lipid nanoparticles, may be formulated to provide immediate and/or modified release of the active agent. Modified release formulations include delayed, sustained, pulsed, controlled, targeted, and programmed release formulations.

### 3. Effective Amounts

The pharmaceutical composition contains an effective amount of the payload component, when present, encapsulated in a polymeric core of the lipid nanoparticles are suitable for preventing, treating, or ameliorating one or more symptoms of a given disease or disorder. As used herein, the terms “and “therapeutically effective amount” mean a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered.

Typically, the amount of the lipid nanoparticles in the pharmaceutical composition is present in an amount effective to treat an inflammatory disease or disorder in a subject e.g., inflammatory bowel disease, ulcerative colitis, or Crohn's disease. In some forms, the pharmaceutical composition is effective to treat inflammation associated with a disease or disorder in a subject e.g., cancer such as colon cancer. In some forms, the pharmaceutical composition is effective to treat inflammation associated with a viral infection in a subject e.g., a coronavirus infection.

Generally, when the lipid nanoparticle contains the payload component e.g., IL-22 mRNA as the core, the total concentration of the payload component in the pharmaceutical composition is in a range from about 0.001 wt % to about 50 wt %, from about 0.001 wt % to about 15 wt %, from about 0.001 wt % to about 10 wt %, from about 0.001 wt % to about 5 wt %, from about 0.001 wt % to about 2 wt %, from about 0.001 wt % to about 0.5 wt %, from about 0.001 wt % to about 0.2 wt %, from about 0.001 wt % to about 0.1 wt %, from about 0.01 wt % to about 20 wt %, from about 0.01 wt % to about 15 wt %, from about 0.01 wt % to about 10 wt %, from about 0.01 wt % to about 5 wt %, from about 0.01 wt % to about 2 wt %, from about 0.01 wt % to about 0.5 wt %, from about 0.01 wt % to about 0.2 wt %, from about 0.01 wt % to about 0.1 wt %, from about 0.1 wt % to about 20 wt %, from about 0.1 wt % to about 15 wt %, from about 0.1 wt % to about 10 wt %, from about 0.1 wt % to about 5 wt %, from about 0.1 wt % to about 2 wt %, from about 0.1 wt % to about 0.5 wt %, or from about 0.1 wt % to about 0.2 wt %. The term “total concentration of the payload component in the pharmaceutical composition” refers to the sum of the weight of all payload components of the lipid nanoparticle relative to the weight of the formulation.

### III. Methods of Use

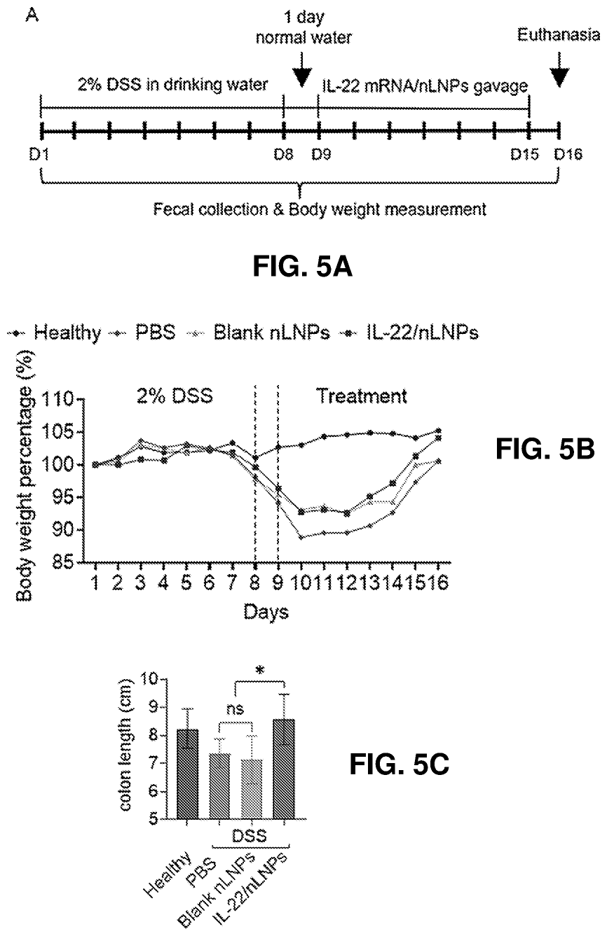

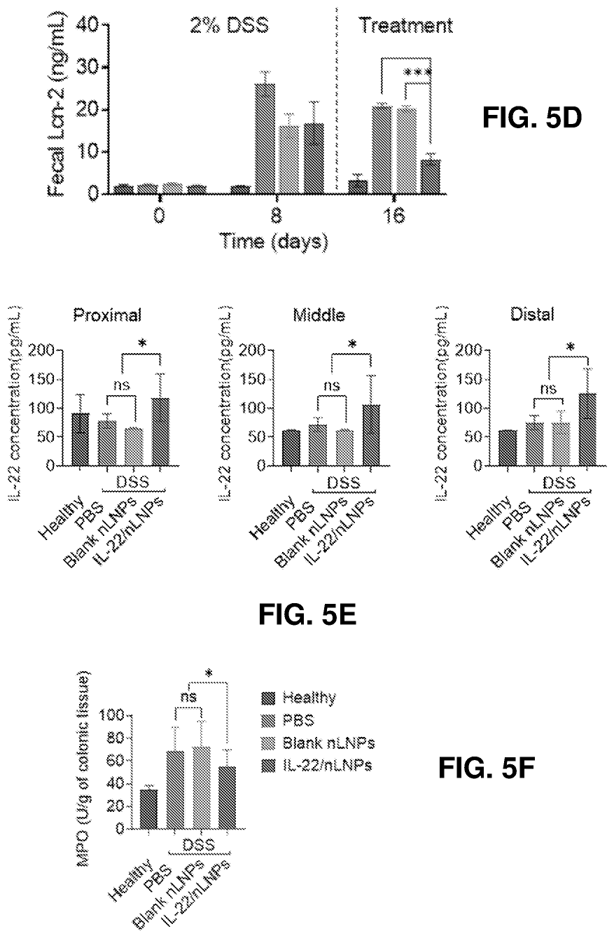

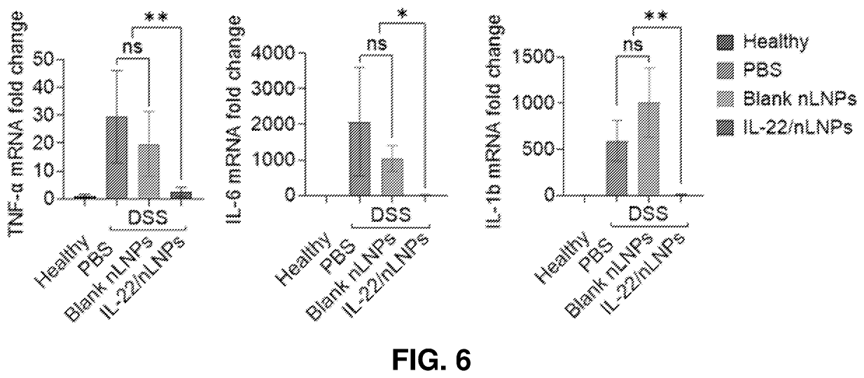

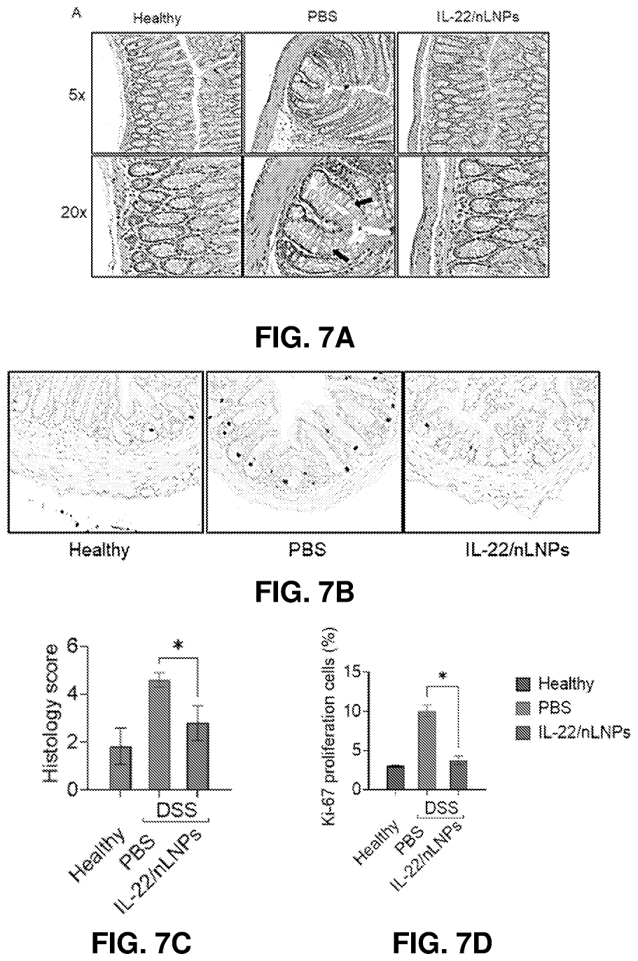

As demonstrated in the non-limiting Examples, administration of IL-22 mRNA-loaded LNPS (or IL-22 LNPs) resulted in an accelerated healing process, as indicated by the recovery of more body weight (see FIG. 5B) and colon length (see FIG. 5C) as well as reduction of the histological index (see FIGS. 7A-7D), colonic MPO activity (see FIG. 5F), fecal lipocalin concentration (see FIG. 5D), and mRNA expression levels of pro-inflammatory cytokines (TNF-α, IL-6, and IL-1β; see FIG. 6). Therefore, also disclosed are methods involving administering the disclosed pharmaceutical compositions to a subject. In some forms, the composition is administered orally to the subject. In some forms, the composition is administered intravascularly to the subject. In some forms, the composition is administered intramuscularly to the subject.

The pharmaceutical compositions containing the lipid nanoparticles, can be used for treating a variety of diseases and disorders such as inflammatory diseases or disorders, cancers and viral infections. It will be appreciated that the disclosed methods can be methods of treatment of microbial infections, inflammatory diseases or disorders, and cancers and the treatment of the symptoms and conditions associated with microbial infections, inflammatory diseases or disorders, and cancers. For example, the pharmaceutical compositions can be administered to a subject in need thereof to prevent or treat an inflammatory disease or disorder in the subject, such as indicated by the improvement or relief of one or more symptoms associated with the inflammatory disease or disorder in the subject.

“Treatment”, as used herein, refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The terms “high,” “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

The term “inhibit” means to reduce or decrease in activity or expression. This can be a complete inhibition of activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “in need of treatment” as used herein refers to a judgment made by a caregiver (e.g., physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that include the knowledge that the subject is ill, or will be ill, as the result of a condition that is treatable by the disclosed compounds and compositions thereof.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

### 1. Treating Inflammatory Diseases or Disorders

The pharmaceutical compositions are suitable for preventing or treating an inflammatory disease or disorder, or treating or ameliorating one or more symptoms associated with an inflammatory disease or disorder in a subject in need thereof. Both acute and chronic inflammatory diseases or disorders can be treated by the disclosed methods. Examples of suitable inflammatory diseases or disorders and symptoms associated with the inflammatory diseases or disorders that can be treated by the disclosed method include, but are not limited to, asthma, chronic peptic ulcer, tuberculosis, rheumatoid arthritis, periodontitis, ulcerative colitis, Crohn's disease, inflammatory bowel disease, sinusitis, active hepatitis, acute bronchitis, appendicitis, ingrown toenail, sore throat, and physical trauma or wound, and a combination thereof. In some forms, the subject is suffering a disease or condition. In some forms, the subject is suffering inflammatory bowel disease. In some forms, the subject is suffering ulcerative colitis. In some forms, the subject is suffering Crohn's disease.

The method for treating an inflammatory disease or disorder in a subject in need of include administering to the subject, a pharmaceutical composition containing a therapeutically effective amount of the lipid nanoparticles to treat the inflammatory disease or disorder, for example, to reduce the severity or prevent one or more symptoms of the inflammatory disease or disorder. The step of administering an effective amount of the pharmaceutical composition can be achieved in a single administration step or using multiple steps of administering the pharmaceutical composition.

### 2. Treating Cancer in a Subject

In some forms, the pharmaceutical compositions containing the lipid nanoparticles can be used in a method for treating cancer in a subject in need thereof. Generally, the method includes administering to the subject, a pharmaceutical composition containing a therapeutically effective amount of the lipid nanoparticles described above, for example, to reduce one or more symptoms of the cancer. The administration step can occur one or more times.

A cancer in a patient refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. In some circumstances, cancer cells will be in the form of a tumor; such cells may exist locally within an animal, or circulate in the blood stream as independent cells, for example, leukemic cells. A tumor refers to all neoplastic cell growth and proliferation, whether malignant or benign, and all precancerous and cancerous cells and tissues. A solid tumor is an abnormal mass of tissue that generally does not contain cysts or liquid areas. A solid tumor may be in the brain, colon, breasts, prostate, liver, kidneys, lungs, esophagus, head and neck, ovaries, cervix, stomach, colon, rectum, bladder, uterus, testes, and pancreas, as non-limiting examples. In some embodiments, the solid tumor regresses or its growth is slowed or arrested after the solid tumor is treated with the presently disclosed methods. In other embodiments, the solid tumor is malignant. In some embodiments, the cancer includes Stage 0 cancer. In some embodiments, the cancer includes Stage I cancer. In some embodiments, the cancer includes Stage II cancer. In some embodiments, the cancer includes Stage III cancer. In some embodiments, the cancer includes Stage IV cancer. In some embodiments, the cancer is refractory and/or metastatic. For example, the cancer may be refractory to treatment with radiotherapy, chemotherapy or monotreatment with immunotherapy. Cancer includes newly diagnosed or recurrent cancers, including without limitation, colitis associated cancer (CAC), acute lymphoblastic leukemia, acute myelogenous leukemia, advanced soft tissue sarcoma, brain cancer, metastatic or aggressive breast cancer, breast carcinoma, bronchogenic carcinoma, choriocarcinoma, chronic myelocytic leukemia, colon carcinoma, colorectal carcinoma, Ewing's sarcoma, gastrointestinal tract carcinoma, glioma, glioblastoma multiforme, head and neck squamous cell carcinoma, hepatocellular carcinoma, Hodgkin's disease, intracranial ependymoblastoma, large bowel cancer, leukemia, liver cancer, lung carcinoma, Lewis lung carcinoma, lymphoma, malignant fibrous histiocytoma, a mammary tumor, melanoma, mesothelioma, neuroblastoma, osteosarcoma, ovarian cancer, pancreatic cancer, a pontine tumor, premenopausal breast cancer, prostate cancer, rhabdomyosarcoma, reticulum cell sarcoma, sarcoma, small cell lung cancer, a solid tumor, stomach cancer, testicular cancer, and uterine carcinoma.

In some forms, the cancer to be treated is a cancer of the small intestine e.g., adenocarcinoma, carcinoid cancer, gastrointestinal stromal tumors (GISTs), lymphoma, sarcoma, or colorectal cancer. In some forms, the cancer to be treated is colon cancer (adenocarcinoma of the colon) and/or rectal cancer. In preferred forms, the cancer to be treated is colon cancer.

The subject can be a mammal, such as a human, a dog, a cat, a rat, a monkey, rabbits, guinea pigs, etc., that is in need of cancer treatment. In some forms, the subject can be exhibiting symptoms of or diagnosed with cancer. A subject in need of treatment includes a subject already diagnosed with a cancer and/or a subject prone to developing a cancer. In some forms, a subject is successfully “treated” for cancer according to the disclosed methods if the patient shows one or more of the following: a reduction in the number of or complete absence of cancer cells; a reduction in the tumor size; inhibition of or an absence of cancer cell infiltration into peripheral organs including the spread of cancer into soft tissue and bone; inhibition of or an absence of tumor metastasis; inhibition or an absence of tumor growth; relief of one or more symptoms associated with the specific cancer; reduced morbidity and mortality; and improvement in quality of life.

In some forms, the methods include administering to a subject with cancer, a pharmaceutical composition containing a therapeutically effective amount of the lipid nanoparticles to slow down, and/or halt progression of a cancer. For example, the amount of lipid nanoparticles administered to the subject is effective to reduce tumor cell viability, slow or halt tumor growth, or to reduce tumor burden in the subject. In some forms, the methods of treating cancer include reducing the tumorigenicity of tumors e.g., by reducing the frequency of cancer stem cells in the tumor. In some forms, the methods can include contacting one or more cancer cells with an effective amount of the lipid nanoparticles, to decrease or inhibit the proliferation and/or viability of the cancer cells compared to untreated control cancer cells.

In some forms, the methods include administering to a subject with cancer, a pharmaceutical composition containing a therapeutically effective amount of the lipid nanoparticles to alter a measurable biochemical or physiological marker. For example, in the case of acute colitis, the amount of the lipid nanoparticles administered to the subject can be effective to reduce the production, inhibit the activation, or inhibit an inflammatory signaling pathway e.g., PI3K/Akt/MTOR, NF-kB, and JAK-STAT pathways. In some forms, the amount of the lipid nanoparticles administered to the subject can be effective to reduce the expression of one or more pro-inflammatory cytokines and/or chemokines including but not limited to TNFα, IL-1β and IL-6.

In some forms, the methods of treating cancer include treating one or more symptoms associated with cancer in a subject. For example, the pharmaceutical compositions containing a therapeutically effective amount of the lipid nanoparticles can be used in a method for prophylactic use i.e., prevention, delay in onset, diminution, eradication, or delay in exacerbation of signs or symptoms after onset, and prevention of cancer relapse. For prophylactic use, a therapeutically effective amount of the lipid nanoparticles and pharmaceutical compositions thereof as described are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms. Prophylactic administration can be used, for example, in the chemopreventative treatment of subjects presenting precancerous lesions, those diagnosed with early-stage malignancies, and for subgroups with susceptibilities (e.g., family, racial, and/or occupational) to particular cancers.

3. Treating Viral Infections and Inflammation associated with Viral Infections

The pharmaceutical compositions are suitable for preventing or treating a viral infections and treating inflammation associated with viral infections.

### 4. Methods of Administration

The methods for treating inflammatory diseases, cancers, and viral infections, or methods for achieving a desired alleviation of symptoms associated with inflammatory diseases, cancers, and viral infections, include administering to an animal, such as a mammal, especially a human being, an effective amount of a combination of a pharmaceutical composition containing a therapeutically effective amount of the lipid nanoparticles and optionally one or more therapeutic, prophylactic or diagnostic agents, such as part of the same formulation, or administered separately and independently at the same time or at different times (i.e., administration of the one or more therapeutic, prophylactic or diagnostic agents, and the lipid nanoparticles is separated by a finite period of time from each other). Therefore, the term “combination” or “combined” is used to refer to either concomitant, simultaneous, or sequential administration of the one or more lipid nanoparticles and one or more optional therapeutic, prophylactic or diagnostic agents. The combinations can be administered either concomitantly (e.g., as an admixture), separately but simultaneously (e.g., via separate intravenous lines into the same subject; one agent is given orally while the other agent is given by infusion or injection, etc.), or sequentially (e.g., one agent is given first followed by the second).

### 5. Effective Amounts

Pharmaceutical compositions containing the lipid nanoparticles and one or more therapeutic, prophylactic, and/or diagnostic agents typically include an effective amount of an admixture of the lipid nanoparticles and one or more therapeutic, prophylactic, and/or diagnostic agents. Effective amounts of pharmaceutical compositions containing the lipid nanoparticles are provided herein. It will be appreciated that in some forms the effective amount of the lipid nanoparticles and one or more therapeutic, prophylactic, and/or diagnostic agents is different from the amount that would be effective for the one or more therapeutic, prophylactic, and/or diagnostic agents to achieve the same result when administered in the absence of the lipid nanoparticles.

When used for treating an inflammatory disease or an inflammatory condition associated with a disease e.g., cancer in a subject, the amount of the lipid nanoparticles present in a pharmaceutical dosage unit, or otherwise administered to a subject, can be the amount effective to reduce the production, inhibit the activation, or inhibit an inflammatory signaling pathway e.g., MAPK signaling. In some forms, the amount of the lipid nanoparticles in a pharmaceutical dosage unit, or otherwise administered to a subject, can be the amount effective to reduce the spread of cancer cells in the colon. Preferably, the amount of lipid nanoparticles present in the pharmaceutical dosage unit, is administered to the subject in an amount effective to reduce the symptoms associated with an inflammatory disease or cancer by about 10%, about 20%, about 30%, about 40%, about 50%, about 60%, about 70%, about 80%, about 90%, or about 99%.

In some forms, the amount of lipid nanoparticles present in a pharmaceutical dosage unit, or otherwise administered to a subject, can be the amount effective to reduce the expression of one or more pro-inflammatory cytokines and/or chemokines including but not limited to TNFα, IL-1β, and IL-6. In some forms, the amount of lipid nanoparticles present in a pharmaceutical dosage unit, or otherwise administered to a subject, can be the amount effective to reduce the expression of one or more genes associated with an inflammatory disease or disorder, cancer, or viral infection in a subject following treatment.

In some forms, the pharmaceutical composition containing the lipid nanoparticles is administered to a subject in need thereof, to deliver the lipid nanoparticle in an amount between about 0.1 mg and about 1000 mg, inclusive, preferably between about 0.5 mg and about 600 mg, inclusive, more preferably between about 1 mg and about 25 mg, inclusive, for example, 2 mg/kg, 5 mg/kg, 10 mg/kg, or 15 mg per kg.

### 6. Dosage Regimens

Dosing regimens are dependent on the severity of the infection or disease/disorder and/or methods of administration, and is known to those skilled in the art. A therapeutically effective amount of the lipid nanoparticles used in the methods of treatment is typically sufficient to reduce or alleviate a viral infection, inflammatory disease/disorder or cancer and symptoms thereof.

A dosage regimen of the pharmaceutical composition containing a therapeutically effective amount of the lipid nanoparticles and optionally one or more therapeutic, prophylactic, and/or diagnostic agents, can include one or multiple administrations of the pharmaceutical formulation.

In some forms, the pharmaceutical compositions are administered at least 1, 2, 3, 4, 5, 6, 7, 8, 9, 10, 11, 12, 13, 14, 15, 16, 17, 18, 19, 20, 21, 22, 23, 24 or 30 hours, or more than 30 hours, up to 36 or 48 hours prior to or after the detection of symptoms associated with an inflammatory disease, cancer, or viral infection in the patient. In other forms, the pharmaceutical compositions are administered at least 1, 2, 3, 4, 5, 6, 7, 8, 9, 10, 11, 12, 13, 14, 15, 16, 17, 18, 19, 20, 21, 22, 23, 24 or 30 hours, or more than 30 hours, up to 36 or 48 hours prior to or after administering a separate therapeutic, prophylactic, or diagnostic agent. In certain forms, additive or more than additive effects of the administration of the pharmaceutical compositions containing the lipid nanoparticles in combination with one or more therapeutic and/or prophylactic agent (s) is evident after one day, two days, three days, four days, five days, six days, one week, two weeks, three weeks, or more than three weeks following administration.

An effective amount of the pharmaceutical compositions and optionally one or more therapeutic and/or prophylactic agents can be administered as a single unit dosage (e.g., as dosage unit), or sub-therapeutic doses that are administered over a finite time interval. Such unit doses may be administered on a daily basis for a finite time period, such as up to 3 days, or up to 5 days, or up to 7 days, or up to 10 days, or up to 15 days or up to 20 days or up to 25 days, are all specifically contemplated.

### 7. Subjects to Be Treated

A subject in need of treatment is a subject having symptoms associated with an inflammatory disease/disorder, cancer or a viral infection, or a subject having or at risk of having an inflammatory disease/disorder, cancer, or a viral infection. Preferably, the subject in need of treatment is a subject having or at risk of having symptoms associated with an inflammatory disease or cancer. In some forms, the subject is a mammal, including, but not limited to, murines, simians, humans, mammalian farm animals and livestock, mammalian sport animals, and mammalian pets. Preferably, the subject is a human.

A subject having an inflammatory disease or disorder is a subject who is experiencing a medical condition characterized by inflammation, which is the body's natural response to harmful stimuli or injury. Inflammatory diseases and disorders can manifest in diverse ways and affect different parts of the body. Inflammation can be localized, confined to a specific area or organ, or it can be systemic, involving multiple organs and tissues throughout the body. Methods of assessing inflammatory diseases/disorders or cancer in a subject are well-established and familiar to those with ordinary skill in the art e.g., blood tests, molecular diagnostics, endoscopy, pathological analysis, and physical examination.

A subject at risk of having cancer is a subject who possesses certain factors or characteristics that increase their likelihood of developing cancer compared to the general population. These risk factors can be genetic, environmental, lifestyle-related, or a combination of these. Example of such subjects include individuals with a family history of certain cancers, carriers of genetic mutations, tobacco users, individuals with chronic inflammatory diseases e.g., inflammatory bowel disease (IBD), chronic hepatitis, or chronic pancreatitis, excessive alcohol consumers, individuals of advanced age and individuals exposed to environmental carcinogens, such as asbestos, benzene, radon, and certain chemicals.

A subject having a viral infection e.g., a coronavirus infection is a subject that has been exposed to a virus and has acute or chronic detectable levels of the virus in his/her body or has signs and symptoms associated with infection of the virus. Methods of assessing and detecting viral infections in a subject are known by those of ordinary skill in the art. A subject at risk of having a viral infection is a subject that may be expected to come in contact with a virus as described above. Examples of such subjects are medical workers or those traveling to parts of the world where the incidence of infection is high. In some forms, the subject is at an elevated risk of an infection because the subject has one or more risk factors to have an infection. Examples of risk factors to be infected and/or develop mild, moderate, and/or severe symptoms include immunosuppression, immunocompromised, age (advanced or very young), and surgery. The degree of risk of infection depends on the multitude and the severity or the magnitude of the risk factors that the subject has. Risk charts and prediction algorithms are available for assessing the risk of an infection in a subject based on the presence and severity of risk factors. Other methods of assessing the risk of infection in a subject are known by those of ordinary skill in the art. In some forms, the subject who is at an elevated risk of an infection may be an apparently healthy subject. An apparently healthy subject is a subject who has no signs or symptoms of disease.

The effect of the pharmaceutical compositions can be compared to a control. Suitable controls are known in the art and include, for example, an untreated subject, or a placebo-treated subject. A typical control is a comparison of a condition or symptom of a subject prior to and after administration of the pharmaceutical compositions including the nanoparticles. The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some forms, the symptom, pharmacologic, or physiologic indicator is measured in a subject prior to treatment, and again one or more times after treatment is initiated. In some forms, the control is a reference level, or average determined based on measuring the symptom, pharmacologic, or physiologic indicator in one or more subjects that do not have the disease or condition to be treated (e.g., healthy subjects). In some forms, the effect of the treatment is compared to a conventional treatment that is known the art. Suitable control subjects are unvaccinated subjects, or subjects receiving the same amount of a therapeutic, prophylactic and/or diagnostic agent in the absence of pharmaceutical compositions containing the lipid nanoparticles.

### 8. Routes of Administration

The lipid nanoparticles and pharmaceutical compositions thereof can be administered in an amount sufficient to ameliorate symptoms associated with an inflammatory disease or disorder, cancer, or viral infection, and are typically administered according to methods known for administering anti-inflammatory therapies to subjects.

In some forms, the lipid nanoparticles and pharmaceutical compositions thereof are administered parenterally. The phrases “parenteral administration” and “administered parenterally” are art-recognized terms, and include modes of administration other than enteral and topical administration, such as injections, and include without limitation intravenous, intramuscular, intrapleural, intravascular, intrapericardial, intraarterial, intrathecal, intratracheal, intranasal intracapsular, intraorbital, intracardiac, intradennal, intraperitoneal, transtracheal, subcutaneous, subcuticular, intraarticular, subcapsular, subarachnoid, intraspinal and intrastemal injection and infusion. The disclosed pharmaceutical compositions can be administered parenterally, for example, by subdural, intravenous, intrathecal, intraventricular, intraarterial, intra-amniotic, intraperitoneal, or subcutaneous routes. In preferred forms, the disclosed pharmaceutical compositions are administered via oral, intravascular, or intramuscular administration. In some forms, the pharmaceutical composition is for use in oral delivery of a drug. In some forms, the pharmaceutical composition is for use in intravascular delivery of a drug. In some forms, the pharmaceutical composition is for use in intramuscular delivery of a drug.

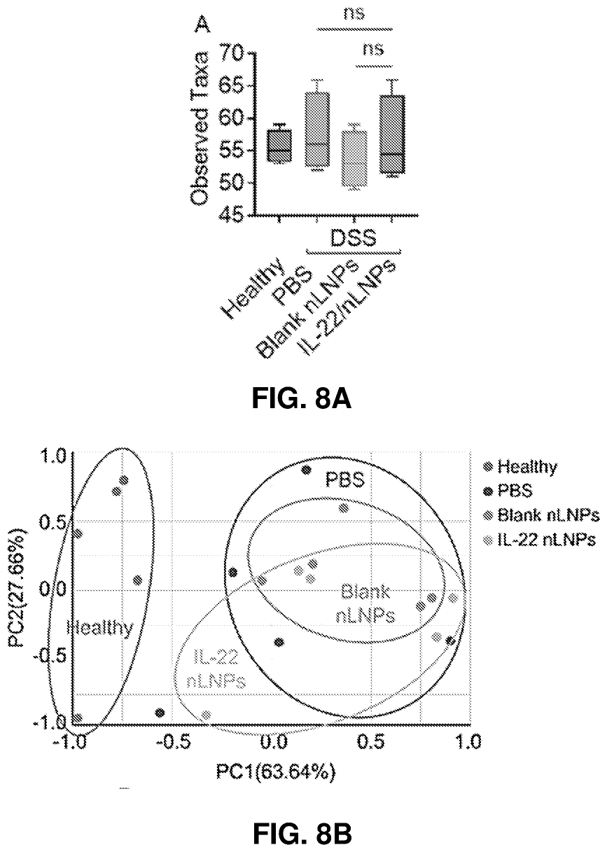

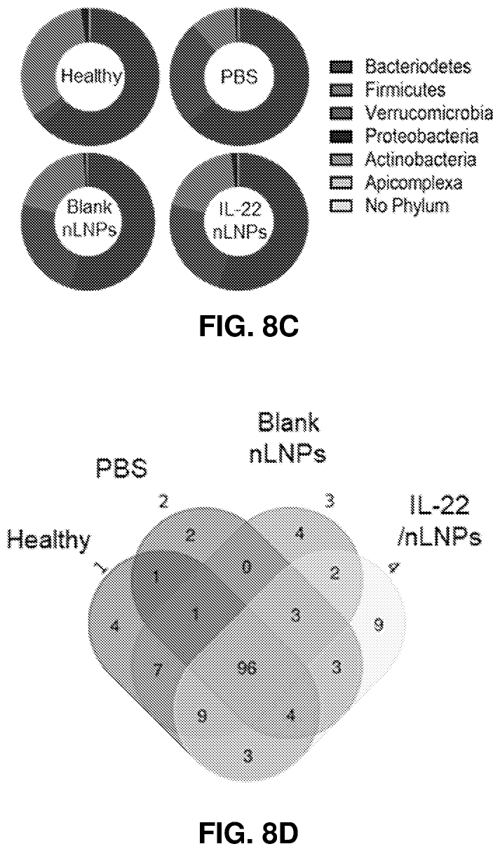

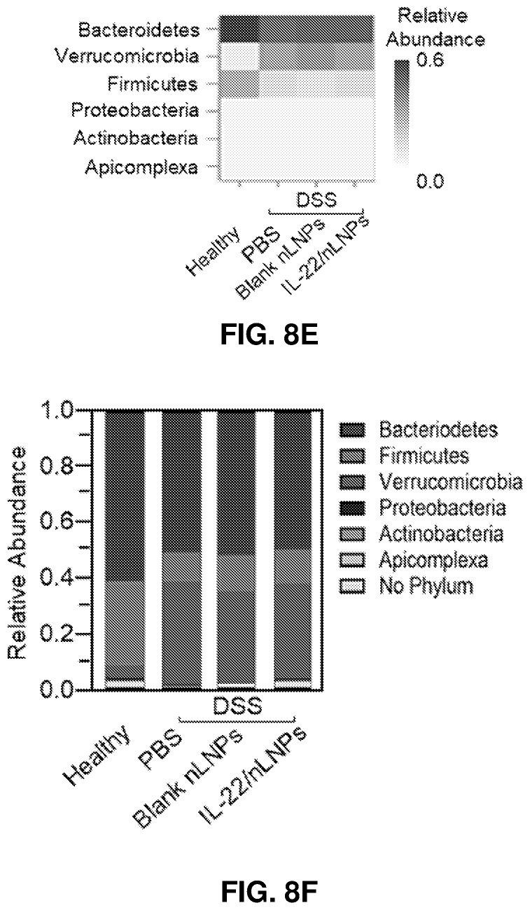

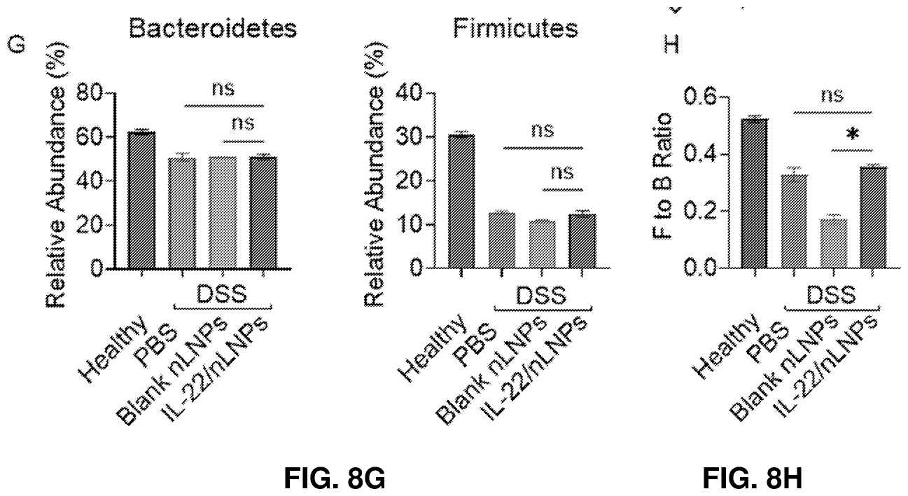

The dosages or amounts of the pharmaceutical compositions described herein, are large enough to produce the desired effect in the method by which delivery occurs. The dosage should not be so large as to cause adverse side effects, such as unwanted cross-reactions, anaphylactic reactions, and the like. For example, the dosage should not be to disrupt the gut microbiota of the intestine. As shown in the non-limiting Examples, the dose of IL-22 NLPs did not disrupt the gut microbiota composition (see FIGS. 8A-8H). Generally, the dosage will vary with the age, condition, sex, and extent of the disease in the subject and can be determined by one of skill in the art. The dosage can be adjusted by the individual physician based on the clinical condition of the subject involved. The dose, schedule of doses and route of administration can be varied.

Also disclosed are drug delivery systems for delivery of a drug, where the drug delivery system includes the disclosed lipid nanoparticles. In some forms, the drug delivery system is formulated for oral delivery. In some forms, the drug delivery system is formulated for intravascular delivery. In some forms, the drug delivery system is formulated for intramuscular delivery.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a nanoparticle is disclosed and discussed and a number of modifications that can be made to a number of components including the nanoparticle are discussed, each and every combination and permutation of nanoparticle and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

The disclosed compositions and methods can be further understood through the following numbered paragraphs.

1. A lipid nanoparticle comprising phosphatidic acid (PA), monogalactosyldiacylglycerol (MGDG), and digalactosyldiacylglycerol (DGDG), wherein the PA, MGDG, and DGDG are present in the nanoparticle in a ratio of 3 to 7, 1 to 3, and 2 to 4, respectively, and wherein the PA, MGDG, and DGDG make up 90% or more of the total lipid in the nanoparticle.

2. The lipid nanoparticle of paragraph 1, wherein the PA, MGDG, and DGDG are each of high purity before being mixed to form the nanoparticle.

3. The lipid nanoparticle of paragraph 1 or 2, wherein no component of the nanoparticle is obtained from ginger.

4. The lipid nanoparticle of any one of paragraphs 1-3, wherein the PA, MGDG, and DGDG are present in the nanoparticle in a ratio of 5:2:3.

5. The lipid nanoparticle of any one of paragraphs 1-4, wherein the PA, MGDG, and DGDG make up 95% or more of the total lipid in the nanoparticle.

6. The lipid nanoparticle of any one of paragraphs 1-5, wherein the PA, MGDG, and DGDG make up 97% or more of the total lipid in the nanoparticle.

7. The lipid nanoparticle of any one of paragraphs 1-6, wherein the PA, MGDG, and DGDG make up 99% or more of the total lipid in the nanoparticle.

8. The lipid nanoparticle of any one of paragraphs 1-7, wherein the PA, MGDG, and DGDG are each of at least 97% purity before being mixed to form the nanoparticle.

9. The lipid nanoparticle of any one of paragraphs 1-8, wherein the PA, MGDG, and DGDG are each of at least 98% purity before being mixed to form the nanoparticle.

10. The lipid nanoparticle of any one of paragraphs 1-9, wherein the PA, MGDG, and DGDG are each of at least 99% purity before being mixed to form the nanoparticle.

11. The lipid nanoparticle of any one of paragraphs 1-10 further comprising ionizable lipids, wherein the ionizable lipids make up 10% or less of the total lipid in the nanoparticle.

12. The lipid nanoparticles of paragraph 11, wherein the ionizable lipids comprise cationic lipids.

13. The lipid nanoparticle of any one of paragraphs 1-12 further comprising one or more surface modifications.

14. The lipid nanoparticle of any one of paragraphs 1-13 further comprising one or more payload components.

15. The lipid nanoparticle of paragraph 14, wherein at least one of the payload components is a compound for delivery to intestine.

16. The lipid nanoparticle of paragraph 14 or 15, wherein at least one of the payload components is a therapeutic agent for treatment of inflammatory bowel disease.

17. The lipid nanoparticle of any one of paragraphs 14-16, wherein at least one of the payload components is a therapeutic agent for treatment of ulcerative colitis.

18. The lipid nanoparticle of any one of paragraphs 14-17, wherein at least one of the payload components is a therapeutic agent for treatment of Crohn's disease.

19. The lipid nanoparticle of any one of paragraphs 14-18, wherein at least one of the payload components is a therapeutic agent for treatment of cancer.

20. The lipid nanoparticle of any one of paragraphs 14-19, wherein at least one of the payload components is a therapeutic agent for treatment of colon cancer.

21. The lipid nanoparticle of any one of paragraphs 14-20, wherein at least one of the payload components is a therapeutic agent for treatment of coronavirus infection.

22. The lipid nanoparticle of any one of paragraphs 14-21, wherein at least one of the payload components is RNA.

23. The lipid nanoparticle of any one of paragraphs 14-22, wherein at least one of the payload components is mRNA.

24. The lipid nanoparticle of any one of paragraphs 14-23, wherein at least one of the payload components is an mRNA encoding IL-22.

25. The lipid nanoparticle of any one of paragraphs 14-24, wherein at least one of the payload components is siRNA or a replicate for siRNA.

26. The lipid nanoparticle of any one of paragraphs 14-25, wherein at least one of the payload components is DNA.

27. The lipid nanoparticle of any one of paragraphs 14-26, wherein at least one of the payload components is pDNA.

28. The lipid nanoparticle of any one of paragraphs 14-27, wherein at least one of the payload components is a protein.

29. A pharmaceutical composition comprising the lipid nanoparticle of any one of paragraphs 1-28.

30. The pharmaceutical composition of claim 29, wherein the composition is formulated for oral administration.

31. A method comprising administering the composition of paragraph 29 or 30 to a subject.

32. The method of paragraph 31, wherein the composition is administered orally to the subject.

33. The method of paragraph 31 or 32, wherein the subject is suffering a disease or condition.

34. The method of any one of paragraphs 31-33, wherein the subject is suffering inflammatory bowel disease.

35. The method of any one of paragraphs 31-34, wherein the subject is suffering ulcerative colitis.

36. The method of any one of paragraphs 31-35, wherein the subject is suffering Crohn's disease.

37. The method of any one of paragraphs 31-36, wherein the subject is suffering cancer.

38. The method of any one of paragraphs 31-37, wherein the subject is suffering colon cancer.

39. The method of any one of paragraphs 31-38, wherein the subject is suffering coronavirus infection.

40. The pharmaceutical composition of paragraph 29 or 30 for use in oral delivery of a drug.

41. The pharmaceutical composition of paragraph 29 or 30 for use in intravascular delivery of a drug.

42. The pharmaceutical composition of paragraph 29 or 30 for use in intramuscular delivery of a drug.

43. A drug delivery system for delivery of a drug, the drug delivery system comprising the lipid nanoparticle of any one of paragraphs 1-28.

44. The drug delivery system of paragraph 43, wherein the drug delivery system is formulated for oral delivery.

45. The drug delivery system of paragraph 43, wherein the drug delivery system is formulated for intravascular delivery.

46. The drug delivery system of paragraph 43, wherein the drug delivery system is formulated for intramuscular delivery.

### EXAMPLES

### Materials and Methods

1.1. Synthesis of IL-22 mRNA Via In Vitro Transcription (IVT)

The full-length IL-22 mRNA (Gene ID: 50929) was designed with an ampicillin-resistant lentiviral vector pMRNA^{xp} Pbackbone, which encodes T7 promoter, 5′UTR, 3′UTR, a polyA tail, and pUC origin (System Bioscience, Palo Alto, CA). With the constructed IL-22 plasmid DNA, in vitro transcription (IVT) method was used to synthesize IL-22 mRNA using the mRNAExpress™ mRNA synthesis kit (System Biosciences, Palo Alto, CA). The cloning mixture containing NTP/Cap mix, reaction buffer, T7 RNA poly mix, nuclease-free water, and template IL-22 plasmid DNA was incubated at 42° C. in the water bath for 2 hrs. Then, the mixture was incubated at 37° C. for 30 min after DNase and phosphatase treatment. DNase was used to digest the DNA template after mRNA synthesis, and phosphatase was used to remove 5′ triphosphates in the final stage of RNA synthesis. Next, newly generated mRNAs were purified by centrifugation with the addition of elution buffer, binding buffer, and 100% ethanol following the manufacturer's instruction (System Biosciences, Cat #MR-KIT-1, ver. 2-070918).

1.2. Preparation and Encapsulation of IL-22 mRNA with Raw Lipid Nanoparticles (rLNPs)

Raw lipids were extracted from ginger-derived nanoparticles (GDNPs) by the liquid-liquid extraction method [7]. After extraction, the upper aqueous phase was removed, and the remaining lower organic phase was dried under vacuum to obtain the raw lipids. Raw lipids were weighed and stored under −20° C. for further use. To make IL-22 mRNA LNPs formulation, prepared was IL-22 mRNA solution in RNase-free water (containing 5% glucose), then, Turbofect (Thermo Fisher Scientific, Waltham, MA) was added mixture was incubated at room temperature for 15 mins. The mixed solution was transferred to a pear-shaped flask with lipid thin film prepared from the raw lipids, and the flask was sonicated with repeated pipetting. Lipid vesicles encapsulated the IL-22 mRNA complex by thin-film hydration. Before the in vivo use, raw lipid nanoparticles (rLNPs) were measured in size and loading efficiency. The detailed procedure can be retrieved from previously published bio-protocol [8].

1.3. Preparation of New Lipid Nanoparticles (nLNPs)

Digalactosyldiacylglycerol (DGDG), Monogalactosyldiacylglycerol (MGDG), and phosphatidic acid (PA) were purchased from Avanti polar lipids (Alabaster, AL). Three lipids were dissolved with dichloromethane (DCM) in glass tubes and mixed them in a ratio of 3:2:5, which mimics the lipid composition discovered in the naturally occurring ginger-derived lipid nanoparticles. Thin-layer chromatography (TLC) was used to demonstrate the lipid profiles from nLNPs and the GDNPs. DGDG, MGDG, and PA standards were also spotted on the TLC plate as references. After spotting the samples, the TLC plate (silica gel, sigma-Aldrich) was developed in a glass chamber with a mobile phase composed of DCM (80%) and Methanol (20%) and visualized by exposing the developed TLC plate to iodine (I₂) vapor. To prepare the nLNPs, the DGDG, MGDG, and PA was transferred mixture to a pear-shaped flask and submitted it to a vacuum Rota-vapor to establish the lipid thin film. The mRNA encapsulation step was identical as described in section 1.2.

### 1.4. Measurement of Particle Size, Zeta Potential, Topographical Morphology, and Loading Efficiency

Lipid nanoparticles were suspended in phosphate-buffered saline (PBS) at a concentration of between 10-100 μg/mL to measure particle size and surface zeta potential. Particle sizes (nm) and zeta potential (mV) of NPs were measured by dynamic light scattering (DLS) using Zetasizer (Malvern Panalytical, Westborough, MA). The average and standard deviations of the diameters (nm) or zeta potential (mV) were calculated using three runs. Each run is an average of 10 measurements. The morphological image was acquired by CoreAFM atomic force microscopy (Nanosurf, Liestel, Switzerland). The detailed procedure can be found in the published bio-protocol paper [9]. Scanning electron microscopy (SEM) images were acquired by Tescan Vega3/EDEX (Tescan Analytics, Fuveau, France).

The loading efficiency (LE) of IL-22 mRNA to nLNPs was tested using the ultracentrifugation method. IL-22/nLNPs (A) or free IL-22 mRNA (B) (at the same concentration) was suspended in 5% glucose with a final concentration of ˜50 μg/mL (calculated by mRNA) and centrifuged using ultracentrifuge Allegra X-15R with TLA 55 rotor (Beckman Coulter, Brea, CA) at 30,000 rpm (55,300×g) and 4° C. for 10 min. The supernatants of A and B were quantitated for mRNA concentrations using a BioTek Synergy 2 plate reader (Agilent, Santa Clara, CA, USA) with an RNA reading plate at UV wavelength 260/280 nm. The LE is calculated by ([absorption of B-absorption of A]/absorption of B)×100%.

### 1.5. In Vitro Stability and In Vivo Biodistribution of IL-22/nLNPs

The stability of new lipid nanoparticles (nLNPs) loaded with IL-22 mRNA was characterized by agarose gel electrophoresis. 10 μL of each sample, including IL-22 mtRNA and nLNP-encapsulated IL-22 mRNA, were loaded into wells of 2% agarose gel with GelRed (Biotium, Fremont, CA) added. Electrophoresis was conducted at 100 V for 45 min in 1×Tris/acetic acid/EDTA buffer (TAE, pH 8) (Biorad, Hercules, CA). The naked IL-22 mRNA and nLNPs encapsulated IL-22 mRNA was exposed to 4 μL of RNase (0.04 mg/mL) at 37° C. for 0, 2, and 4 hrs. The migrated samples were analyzed under the chemiluminescence detector ChemiDoc XRS+(Biorad, Hercules, CA). Further, the stability of IL-22 mRNA and IL-22/nLNPs in both simulated intestinal fluids (SIF) and simulated gastric fluids (SGF) was evaluated. The IL-22/nLNPs were incubated in SGF or SIF for 4 hrs, respectively. Then the samples were collected, and electrophoresis was used to determine whether IL-22 mRNA is protected against the gastric condition. For in vivo biodistribution study, the IL-22/nLNPs were labeled with lipophilic fluorescent cyanine dye, DiR′, and administered orally in mice for 7 consecutive days. Then the mice were euthanized to measure the biodistribution of DiR′-labeled IL-22/nLNPs in different organs, including the stomach, small intestine, colon, heart, liver, spleen, lung, kidney, and brain. Blood samples were also collected to measure the IL-22 protein concentrations in the serum (ELISA) after 7-day treatment.

1.6. In Vitro Transfection of IL-22 Plasmid DNA (pDNA) and mRNA

Colonic epithelial cells (Caco2-BBE, ATCC, Manassas, VA) were used to transfect IL-22 pDNA and mRNA. Approximately 5×10⁴ cells were seeded on a 24-well plate prefilled with 1 mL Dulbecco's modified eagle medium (DMEM) per well. When the cells reached 80% confluency, 1 μg of plasmid DNA/mRNA and 2 μl of Turbofect (Thermo Fisher Scientific, Waltham, MA) were mixed and diluted in 100 μl serum-free media and incubated at for 15 min at room temperature, then the diluted mixtures were added to the 0.9 mL of DMEM media. Samples were pipetted dropwise to each well and the plates were gently rocked. Supernatants were collected after 48, 72, and 96 hrs post-transfection to measure the level of IL-22. In addition, GDNPs- and nLNPs-encapsulated mRNA were used to compare their IL-22 mRNA delivery efficiency. Caco2-BBE cells were incubated with GDNPs- or nLNPs-encapsulated IL-22 mRNA and the expression levels of IL-22 were measured by ELISA after 72 h.

1.7. In Vivo Expression of IL-22 Plasmid DNA (pDNA) or mRNA in Healthy Mice

C57BL/6 mice (female, 6 to 8-week-old) were purchased from Jackson Laboratory (Bar Harbor, ME) and acclimatized for a week in the animal facility, with 12 h light and dark cycles. Mice were divided into two groups (3 mice in each group; Group 1: pDNA/nLNPs, Group 2: mRNA/nLNPs). All mice were fasted for 4 h before gavage and for 1 h after gavage. Group 1 mice were orally delivered with IL-22 pDNA-loaded nLNPs and group 2 mice were orally delivered with IL-22 mRNA-loaded nLNPs daily for 3 days. Dosed volume for each mouse was 200 μL and the dosage was 200 μg/kg of mRNA (4 μg mRNA/mouse) or 400 μg/kg of pDNA (8 μg pDNA/mouse). Mice were euthanized 4 days after being gavaged with pDNA-loaded nLNP and 1 day after being gavaged with mRNA-loaded nLNP treatment, respectively. Colon tissue samples were separately collected from the proximal, middle, and distal regions and homogenized (a detailed method can be found in section 1.9). Tissue extracts were acquired by centrifugation, and IL-22 protein expression levels was measured in three parts of the colon (proximal, middle, and distal) following a protocol for the quantitative mouse ELISA kit (Abcam, Cambridge, UK).

The full length sequence of IL-22 plasmid DNA is shown as SEQ ID NO: 1.
IL-22 sequence (Organism: Mus musculus, Gene ID: 50929)
(SEQ ID NO: 1)
CATGGCTTTCTTTGAAAACTGTGTTTAGAAGATTTCTGGGATTTGTGTGCAAA
AGCACCTTGTTGGCCCTCACCGTGACGTTTTAGGGAAGACTTCCCATCTCTCA
AGGTGGGAAGGCTTGGAGGTGGTGTCTTGTGGCCTCCTATGGTGGTTAGGTA
CTTCTCAGAAGACAGGACTGGAAATTAGATAATGTCTGATGTCATATCATTC
ACAATACCAAAAAAACCCTGGTGTCCCGATGGCTATAAAAGCAGCAACTTCT
GCCTCTCCCATCACAAGCAGAGACACCTAAACAGGCTCTCCTCTCAGTTATC
AACTGTTGACACTTGTGCGATCTCTGATGGCTGTCCTGCAGAAATCTATGAGT
TTTTCCCTTATGGGGACTTTGGCCGCCAGCTGCCTGCTTCTCATTGCCCTGTG
GGCCCAGGAGGCAAATGCGCTGCCCGTCAACACCCGGTGCAAGCTTGAGGT
GTCCAACTTCCAGCAGCCATACATCGTCAACCGCACCTTTATGCTGGCCAAG
GAGGCCAGCCTTGCAGATAACAACACAGATGTCCGGCTCATCGGGGAGAAA
CTGTTCCGAGGAGTCAGTGCTAAGGATCAGTGCTACCTGATGAAGCAGGTGC
TCAACTTCACCCTGGAAGACGTTCTGCTCCCCCAGTCAGACAGGTTCCAGCC
CTACATGCAGGAGGTGGTGCCTTTCCTGACCAAACTCAGCAATCAGCTCAGC
TCCTGTCACATCAGCGGTGACGACCAGAACATCCAGAAGAATGTCAGAAGG
CTGAAGGAGACAGTGAAAAAGCTTGGAGAGAGTGGAGAGATCAAGGCGATT
GGGGAACTGGACCTGCTGTTTATGTCTCTGAGAAATGCTTGCGTCTGAGCGA
GAAGAAGCTAGAAAACGAAGAACTGCTCCTTCCTGCCTTCTAAAAA.

1.8. In Vivo Delivery of IL-22 mRNA in Acute Colitis Mice

A total of 20 C57BL/6 mice (female, 6 to 8-week-old, Jackson Laboratory) were acclimatized for a week in the animal facility, with 12 h light and dark cycles. Mice were evenly divided into 4 groups (5 mice in each group; Group 1: Healthy control, Group 2: DSS+PBS, Group 3: DSS+blank nLNPs, and Group 4: DSS+IL-22/nLNPs). Groups 2-4 were treated with 2% DSS (MP Biomedicals, Santa Ana, CA) for 7 days in a row to induce mild colitis. Before treatment, mice were rested for a day with normal water. All mice have fasted 4 h before gavage and 1 h after gavage. Then, water, PBS, nLNPs, and IL-22/nLNPs were gavaged daily for another 7 days to observe the recovery status of each group. The dosage of IL-22/nLNPs was 200 μg/kg and dosed volume was 200 μL for each mouse. Body weights were measured every day, and fecal samples were collected every 2 days and stored at 80° C. for further inflammatory parameter analysis. All the mice were euthanized 24 h after the last treatment. Organs (colon and spleen) were collected, and colon length and spleen weight were measured prior to storage (at −80° C.). Colon tissues were cut into three parts: proximal, transverse, and distal. All experimental procedures involving animals were approved by the Institutional Animal Care and Use Committee (IACUC, Georgia State University, Atlanta, GA, No. A20039) and complied with ethical regulations for animal testing and research.

### 1.9. Measurement of IL-22 Expression in Mouse Colon

Colon tissues were placed in 2 mL Beadbug prefilled microtubes with 2 mm triple-pure high-impact Zirconium beads (Millipore Sigma, Burlington, MA) and homogenized in 1×RIPA lysis and extraction buffer (Thermo Fisher Scientific, Waltham, MA) including protease inhibitor (Roche, Basel, Switzerland) with Beadblaster 24 (Benchmark Scientific, Sayreville, NJ). The homogenizing program was set as 6.0-speed m/s, 45-see shaking, and 15-see intermissions for 4 cycles at 4° C. Microtubes containing homogenized colon tissues were centrifuged at 10,000×g for 20 min at 4° C. to pellet the debris and then carefully transferred the clear supernatants to new 1.5 mL tubes without any disturbance of pellets. Collected supernatants were used to measure the quantitative level of IL-22 protein expression in the colon. Prepared 96 well ELISA plates loaded with samples and capture antibody/detector antibody mixture was incubated on a tabletop shaker (Thermo Fisher Scientific, Waltham, MA) for 1 hr at room temperature. Plates were washed well 3 times with wash buffer, and 3,3′,5,5′-tetramethylbenzidine (TMB) development solution was added and then incubated in the dark for 10 min on the shaker again. After the blue color developed, a stop solution was added to measure OD at 450 nm with a multi-detection microplate reader Synergy 2 (BioTek, Winooski, VT).

### 1.10. Colonic Myeloperoxidase (MPO) Assay

Pre-weighed colon tissues (50 mg/mL) were washed with PBS and homogenized in 0.5% hexadecyltrimethylammonium bromide (HTAB). Sonication was followed up at 40% amplitude, 5 sec on/off. Three cycles of freezing and thawing were done at −80° C. and 37° C. conditions. Then, the samples were centrifuged at 14,000 rpm for 15 min at 4° C. Clear supernatants and MPO standards were loaded on 96 well plates, and reactive buffers were added to each well immediately after preparing the solutions. After colors developed, optical density was read at 450 nm.

### 1.11. Measurement of Fecal Lipocalin-2 (Lcn-2)

Pre-weighed fecal samples were homogenized by vortex for 15 min, and supernatants were collected after centrifugation at 14,000×g, 4° C. for 15 min. Samples and standards were loaded on capture antibody pre-coated 96-well plates and incubated for 2 h at room temperature. After 3 washing steps, detection antibody, streptavidin-HRP, and substrate solution were added, respectively. Complete aspiration of each solution was done between the procedures. Optical density was read at 450 nm and 540 nm immediately after adding the stop solution. Readings were subtracted from 450 nm to 540 nm to correct imperfect data recording.

1.12. Quantitation of Colonic Cytokine mRNA Expression

Colon extracted and purified total RNA by RNeasy Mini Kit (Qiagen, Hilden, DE) was used to generate complementary DNA (cDNA) with Maxima cDNA Synthesis Kit (Thermo Scientific, Waltham, MA). Maxima SYBR Green/ROX qPCR Master Mix (Thermo Scientific, Waltham, MA) was used to analyze the RNA expression level of pro-inflammatory cytokines (TNF-α, IL-6, and IL-1b) and anti-inflammatory cytokines (IL-10 and IL-22) with 96-well format thermal cycler (Eppendorf, Hamburg, Germany). The housekeeping gene, 36B4, was used as a normalization cytokine. Following sense and anti-sense primers were used: TNF-α: 5′-AGG CTG CCC CGA CTA CGT-3′ (SEQ ID NO:2) and 5′-GAC TTT CTC CTG GTA TGA GAT AGC AAA-3′ (SEQ ID NO:3); IL-1b: 5′-TTG ACG GAC CCC AAA AGA TG-3′(SEQ ID NO:4) and 5′-AGA AGG TGC TCA TGT CCT CAT-3′(SEQ ID NO:5); IL-6: 5′-ACA AGT CGG AGG CTT AAT TAC ACA T-3′ (SEQ ID NO:6) and 5′-TTG CCA TTG CAC AAC TCT TTT C-3′ (SEQ ID NO:7); IL-10: 5′-GGT TGC CAA GCC TTA TCG GA-3′ (SEQ ID NO:8) and 5′-CTT CTC ACC CAG GGA ATT CA-3′(SEQ ID NO:9); IL-22: 5′-GTC AAC CGC ACC TTT ATG CT-3′ (SEQ ID NO:10) and 5′-GTT GAG CAC CTG CTT CAT CA-3′(SEQ ID NO:11); and 36B4: 5′-TCC AGG CTT TGG GCA TCA-3′ (SEQ ID NO:12) and 5′-CTT TAT CAG CTG CAC ATC ACT CAG A-3′(SEQ ID NO:13). Data were recorded and analyzed by Mastercycler ep Realplex version 2.2 (Eppendorf, Hamburg, Germany).

1.13. Immunohistochemical Analysis after IL-22 mRNA Treatment

Colonic tissue samples were fixed in 10% buffered formaldehyde overnight at 4° C. and stored in 70% EtOH. Samples were embedded in paraffin block to be sectioned and mounted on glass slides for staining. A standard H&E staining method was used. Apoptosis and proliferation were examined by immunohistochemical analysis using TUNEL and Ki-67, respectively. Primary antibodies were incubated with deparaffinized sectioned samples for 1 hr at room temperature. Sections were then incubated with secondary antibodies for 30 min.

### 1.14. Fecal Microbiome Analysis by Whole-Genome Sequencing (WGS)

Fresh mouse fecal samples (two pieces for each mouse) were collected and immediately immersed in the Transnetyx microbiome sample collection tubes (1.5 mL, prefilled with 0.8 mL RNA stabilization liquid and glass beads). Then, all the samples were submitted to the Transnetyx sequencing platform, which leverages shallow shotgun WGS with a minimum read depth of 2 million paired-end reads for the methodology and provides species/sub-strain level taxonomic resolution on each sample. The analysis includes all microbe types, such as bacteria, viruses, fungi, protists, and archaea. Data visualization of taxonomy, alpha-, beta-diversity, clustering, and PCoA were performed at the Onecodex (onecodex.com) cloud computing website.

### 1.15. Blood Counting and Blood Chemistry Assay

Blood was collected from the retro-orbital sinus of the mice and all mice were euthanized after the blood collection, according to the animal protocol (No. A20039, IACUC, Georgia State University, Atlanta, GA). EDTA-K3 coated tubes (1.3 mL, K3E, Sarstedt AG & Co. Germany) were used to store blood for further hematological analyses and Li/Heparin coated tubes (1.3 mL, LH, Sarstedt AG & Co. Germany) were used to store blood for further blood chemistry test. Blood counting was performed using an automatic hematology analyzer (VetScan HM5; Abaxis, CA, USA). 50-100 μL of fresh blood was used for each test and the following hematologic parameters were tested: WBC, total white blood cells; LYM, lymphocytes; MON, monocytes; NEU, neutrophils; RBC, red blood cells; HGB, hemoglobin; HCT, hematocrit; MCV, mean corpuscular volume; MCH, hemoglobin amount per red blood cell; MCHC, mean corpuscular hemoglobin concentration; RDWc, red cell distribution width cv; RDWs, red cell distribution width; PLT, platelets; MPV, mean platelet volume; PCT, procalcitonin; PDWc, Platelet distribution width cv; PDWs, Platelet distribution width. Blood biochemistry analysis was performed using 100 μL of fresh blood from each mouse. An automatic hematology analyzer (VetScan VS2; Abaxis, CA, USA) with ABAXIS comprehensive diagnostic rotor (ABAXIS Europe GmbH, Germany) was employed, which can quantitate ALT, alanine aminotransferase; ALB, albumin; ALP, alkaline phosphatase; AMY, amylase; TBIL, total bilirubin; BUN, urea nitrogen; CA, calcium; PHOS, phosphorus; CRE, creatinine; GLU, glucose; NA+, sodium; K+, potassium, TP, total protein; and GLOB, globulin.

### 1.16. Data and Graphic Analysis

Microsoft excel 2013 was used for data recording and processing. GraphPad Prism 9.01 was utilized for data analysis and visualization. All the data presented are biological replicates, and the outliers were calculated and removed by the outlier calculator in GraphPad with an alpha value of 0.05. Significance was determined using unpaired two-tailed Student's t-test and differences were noted as significant (*p<0.05, **p<0.01, ***p<0.001, and ****p<0.0001).

### Results

2.1. Synthesis of In Vitro Transcribed IL-22 mRNA

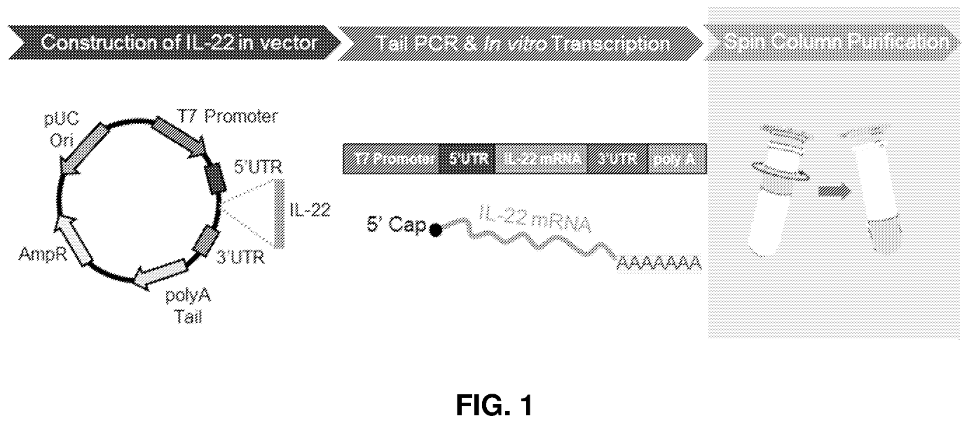

In vitro transcription (IVT), which is a simple method for cDNA template-directed mRNA synthesis, was introduced by Gelder et al. in 1990 [10]. FIG. 1 demonstrates the simplified steps used to generate the synthetic IL-22 mRNA via IVT. First, a plasmid DNA (pDNA) was designed containing the targeted IL-22 gene. The constructed pDNA was affixed with a 3′ poly-A tail to increase the stability and regulate the translational activity of mRNA. The attached poly-A tail is critical for the ability of the sequence to interact with poly-A binding protein (PABP) and also protects the mRNA from 3′ to 5′ nuclease degradation [11]. The purified template DNA was then used to perform IVT with bacteriophage T7 RNA polymerase and Cap analogs. The original DNA template was digested by DNase, and the 5′ triphosphate group was removed by phosphatase to help prevent possible immune responses. Filtered centrifugation was used to remove unreacted nucleotides, remaining enzymes, and residual salts to obtain high-quality synthetic IL-22 mRNA. The final concentration of the purified synthetic IL-22 mRNA was approximately 0.5-1 mg/mL.

2.2. IL-22 mRNA-Loaded nLNPs (IL-22/nLNPs) Prevent the Degradation of IL-22 mRNAs

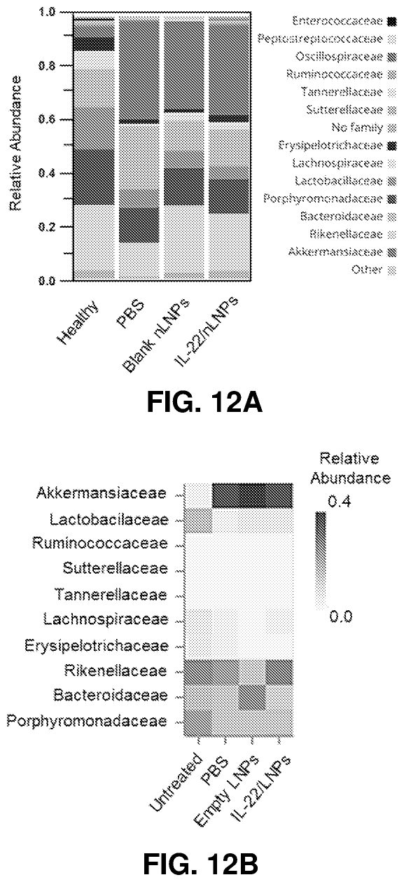

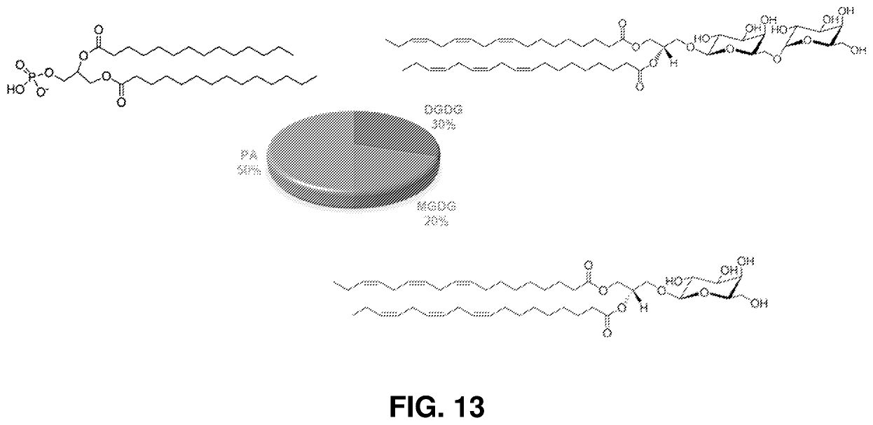

Lipid nanoparticles have shown a robust capability to encapsulate and deliver variously sized molecules to desired regions in animal models [12, 13]. Having previously identified the lipid compositions of ginger-derived nanoparticles (GDNPs) [8, 14], a simplified lipid nanoformulation containing digalactosyldiacylglycerol (DGDG), monogalactosyldiacylglycerol (MGDG), and phosphatidic acid (PA) was used to generate nLNPs (FIG. 12), loaded them with the desired mRNA (that encoding IL-22) with the help of a cationic polymer (turbofectamine, to stabilize and condense the mRNA within the nanoparticles), and assessed the ability of the generated particles to protect the desired mRNA.

The particle size and surface zeta potential of the generated mRNA-loaded nLNPs was checked using a dynamic light scattering device. The average diameter of the nLNPs was ˜200 nm, and the zeta potential was −29±0.5 mV (FIG. 2A-B). Also, the size and zeta potential of both nLNPs and IL-22/nLNPs were maintained in the PBS solution for more than 5 hrs (FIG. 2F). Thin-layer chromatography (TLC) showed clear separation of nLNPs components, including DGDG, MGDG, and PA. Also, the TLC lipid profile of nLNPs is similar to the profile of GDNPs (FIG. 2C, right two columns). Scanning electron microscopy (SEM) was used to examine the morphology of nLNPs. They appeared to have a generally spherical shape with a particle size of 200 nm (FIG. 2D). Atomic force microscopy (AFM) also visualized the spherical morphology of nLNPs. These parameters are similar to those of the original GDNPs [7].

To determine the load efficiency (LE) of mRNA to nLNPs, quantitated was the IL-22 mRNA concentrations before and after loading it to nLNPs. The result shows that nLNPs could yield 67-74% of mRNA loading, which is excellent for the encapsulation of a hydrophilic biomolecule such as mRNA (FIG. 14). Next, to test whether nLNPs could effectively protect the encapsulated mRNA, agarose gel electrophoresis was performed using free IL-22 mRNA, RNase-treated IL-22 mRNA, and RNase-treated nLNP-encapsulated IL-22 mRNA. Gel imaging showed that free IL-22 mRNA was degraded by RNase, whereas nLNP-encapsulated IL-22 mRNA was not (FIG. 2E). In addition, free IL-22 mRNA and IL-22/nLNPs were incubated in simulated gastric fluid (SGF) or simulated intestinal fluid (SIF) for 4 hrs to test whether encapsulated IL-22 mRNA could survive in the GI tract during transportation. While free IL-22 mRNA is unstable in SGF and SIF, nLNPs were able to prevent most of the encapsulated mRNA from degrading in SGF and SIF. These findings suggest that nLNPs may protect encapsulated IL-22 mRNAs and thus might be able to deliver this mRNA to the target region.

2.3. IL-22 mRNA is More Efficient than IL-22 pDNA for Expressing IL-22 Protein in Intestinal Epithelial Cells In Vitro

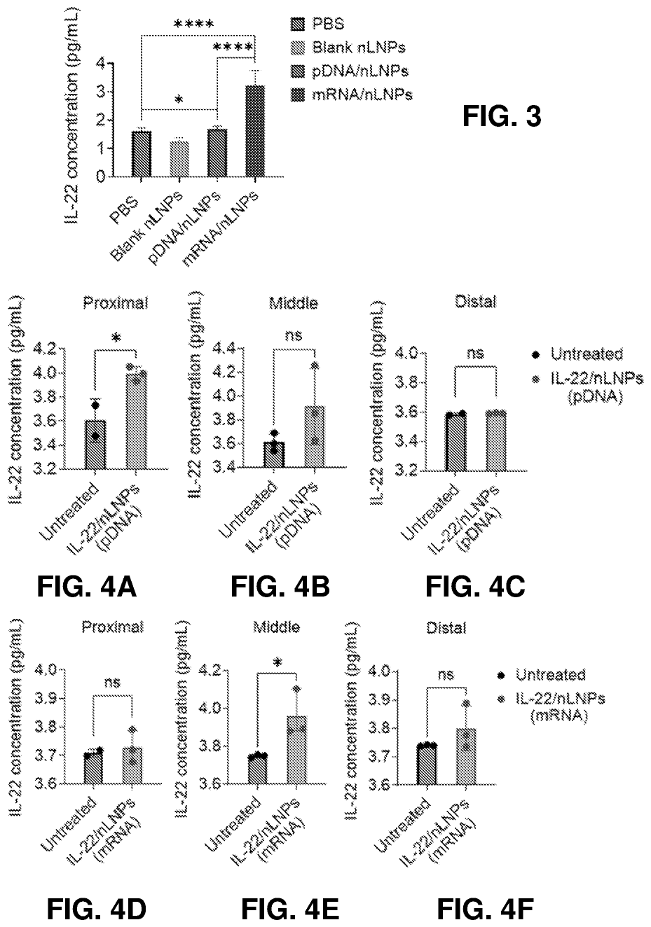

The major expected advantage of mRNA over DNA for gene delivery is that mRNA has a higher transfection efficiency: mRNA can directly enter the cytoplasm to undergo immediate translation, whereas pDNA must first cross the nuclear membrane to undergo transcription. To compare the transfection efficiency of pMRNAxp-IL-22 mRNA and CD813-IL-22 pDNA, nLNPs loaded with each formulation to Caco-2 BBE cells under identical culture and treatment conditions were transfected, collected the cell culture medium after 72 h of incubation, and measured the level of IL-22 by ELISA. As shown in FIG. 3, cells transfected with IL-22 mRNA-loaded nLNPs generated over 3 pg/mL of IL-22 protein, whereas those transfected with pDNA-loaded nLNPs showed only a slight increase to 1.8 pg/mL. This demonstrates that the nLNP-mediated delivery of IL-22 mRNA is far more efficient than that of IL-22 pDNA in increasing the protein expression of IL-22 in vitro. The transfection efficiency was also compared between GDNPs- and nLNPs encapsulated mRNA formulations. The data showed that the transfection efficiency of reversely-engineered nLNPs was similar to that of GDNPs (FIG. 15).

### 2.4. Oral Delivery of IL-22/nLNPs to the Colon Induces Colonic IL-22 Protein Expression

As mRNA is easily degraded in the GI tract, the main function of a colon-targeting delivery system must be to maintain the stability of mRNA when passing through the GI tract. Previous studies demonstrated that orally delivered GDNPs could effectively escort encapsulated drugs and siRNAs to the colon [14], indicating that GDNPs maintain their nanostructure while transiting the GI tract. LC-MS/MS-based lipid analysis showed that the lipid composition of the GDNPs nanostructure is DGDG, MGDG, and PA, and that they occur in a respective ratio of 3:2:5 [15]. The three lipids together were found to make up more than 90% of the lipids in colon-targeting GDNPs. Here, the same ratio was used to construct well-defined nLNPs that contained only these three major lipids. After thin-film hydration, the constructed nLNPs were found to have characteristics (such as morphology, size, and zeta potential) nearly identical to those of the original colon-targeting GDNPs.

Given that a quick increase of IL-22 in the intestinal mucosa can accelerate the resolution of inflammation [16] whereas prolonged IL-22 upregulation may link to colitis-associated colon cancer, it was investigated whether oral delivery of nLNP-encapsulated IL-22 mRNA to healthy mice could boost their intestinal protein expression of IL-22 much quicker compared to oral delivery of nLNP-loaded IL-22 pDNA. In this context, both IL-22 mRNA and pDNA were administered 3 days in a row, mRNA-treated mice were euthanized 1-day post-treatment whereas the pDNA group was euthanized 4 days after the treatment. As seen in FIG. 4, both IL-22 mRNA and pDNA altered the colonic IL-22 expression but in a different pattern. Importantly, IL-22 mRNA was able to achieve the same degree of effect in just one day after treatment. The different efficacy pattern yield from IL22 mRNA and pDNA is an observation that requires more investigation. These findings suggest that oral delivery of IL-22/nLNPs could be a promising approach for instantly inducing colonic IL-22 protein expression.

### 2.5. Oral Administration of IL-22/nLNPs Accelerates Healing in a Mouse Model of Intestinal Wound Healing

After confirming that IL-22/nLNPs could instantly increase colonic IL-22 expression in healthy mice, it was tested whether this enhancement could expedite the healing of acute colitis. The DSS-induced mouse model of acute colitis was employed, wherein DSS causes epithelial lining loss and mucosal barrier damage to model human IBD. DSS was administered for the first 7 days to induce mild colitis, and regular water was administered for 1 day to allow the mice to rest (FIG. 5A) and reduce the risk that the nLNPs would be disturbed by any DSS remaining in the GI tract [17], and then IL-22/nLNPs, blank nLNPs, or PBS were orally administered for another 7 days. Fecal samples and body weight data were also collected every 2 days throughout the experiment. At the end of this treatment (day 15), it was found that IL-22/nLNP-treated mice had recovered their body weight to near that of the healthy mouse group (which received only water and showed a natural increase in body weight over time), whereas the PBS and blank nLNP groups showed significantly lesser recovery of body weight than IL-22/nLNP-treated group. (FIG. 5B).

It was also investigated colon length and spleen weight as additional inflammatory parameters. The average colon length of the IL-22/nLNP-treated group was greater than those of the PBS- and blank nLNP-treated groups (FIG. 5C), whereas the spleen weight did not differ significantly between groups (FIG. 16). Stool samples were collected during the treatment and the fecal lipocalin-2 (Lcn-2) level was measured, which is a sensitive and non-invasive biomarker for intestinal inflammation in murine models. As shown in FIG. 5D, 7-day 2% DSS treatment was associated with a significant increase of fecal Lcn-2 in all groups, compared to the healthy control group. On day 16, the Lcn-2 level was dramatically decreased (>50%) in the IL-22/nLNP group, but not significantly altered in the PBS and blank nLNP groups. These findings suggested that oral administration of IL-22 mRNA-loaded nLNPs significantly speeds up the healing phase in a mouse model of intestinal wound-healing.

After the 7-day IL-22/nLNP treatment, it was expected that IL-22 expression would be upregulated in the colon of DSS-induced colitis mice. IL-22 in the proximal, middle, and distal colon was measured. The results revealed that the concentrations of IL-22 protein in all sections were significantly increased after IL-22/nLNP treatment (FIG. 5F). Neutrophil accumulation in the inflamed intestinal mucosal layer is a prominent feature in UC. Inflammatory severity is often correlated with colonic myeloperoxidase (MPO) activity, representing neutrophil accumulation into the mucosa during intestinal inflammation can be measured. It was found that the MPO activity of the IL-22/nLNP-treated group was significantly lower than those of the PBS or blank nLNP groups (FIG. 5E), indicating that the nLNP-delivered IL-22 also reduced the inflammation associated with neutrophil accumulation.

### 2.6. Oral Delivery of IL-22/nLNPs Suppresses Colonic Expression of Pro-Inflammatory Cytokines

Several cytokines are known to orchestrate the initiation, evolution, and resolution of pathophysiological progress in UC, and their concentrations are taken as crucial indicators of disease severity [4]. Therefore, the expression levels of the critical pro-inflammatory cytokines, TNF-α, IL-1β, and IL-6 were examined. Studies have shown that TNF-α exerts its pro-inflammatory effects by amplifying the downstream cytokines, IL-1β and IL-6, which subsequently modulate adhesion molecule expression, fibroblast proliferation, procoagulant factors, acute-phase cytotoxic/apoptotic responses, and general apoptosis ┌5,6┐. It was found that oral administration of IL-22 mRNA-loaded nLNPs downregulated IL-1β, IL-6, and TNF-α (FIG. 6), which have been implicated in the inflammatory responses responsible for intestinal mucosal damage [18, 19].

2.7. Histological Analysis Shows that Oral Administration of IL-22/nLNPs Accelerates the Healing of Injured Intestinal Mucosa

Histological analysis is a powerful tool for evaluating the level of inflammation in damaged colonic tissues. Three main histological parameters can be taken as reflecting the severity of inflammation: morphological changes of crypts, inflammatory cell infiltration, and overall mucosal structure [7]. It was examined whether orally administered IL-22/nLNPs could have therapeutic impacts on these histological scores. As shown in FIG. 7A, healthy control and IL-22/nLNP-treated colon tissues displayed well-defined epithelial and no leukocyte infiltration in the mucosa, whereas the PBS group showed evidence of severe colonic damage, such as inflammatory cell infiltration, crypt distortion, and epithelial thickening. As a result, the histology score of the IL-22/nLNP-treated group was significantly lower than that of the PBS group (FIG. 7C).

2.8. Oral Administration of IL-22/nLNPs does not Affect the Gut Microbial Composition

The gut microbiota supports the physiological and immunological functions of the GI tract by interacting with the intestinal mucosal barrier and modulating host metabolism and immunity [8,9]. To study the impact of orally delivered IL-22 mRNA-loaded nLNPs on the gut microbiota, fecal samples from control and treatment groups were collected and whole-genome sequencing (WGS) was used to investigate their gut microbiota composition. The taxonomic profile and microbial composition of each group at the phylum level was compared. The results indicated that oral administration of IL-22/nLNPs did not noticeably impact the intestinal microbiota composition. The observed taxonomy and species diversity (Shannon index, Simpson index) showed no significant difference between the groups with or without IL-22/nLNP treatment (FIG. 8A). The similarities and differences of the groups were visualized and compared by conducting a principal coordinate analysis (PCoA) (FIG. 8B). Each colored dot represents an individual mouse (green, untreated; purple, PBS; navy, blank nLNPs; and orange, IL-22/nLNPs), and the distance between each group represents the degree of difference. While the gut microbiota of DSS-treated mice was distinct from those of healthy mice even after the 7-day recovery phase, those of the nLNP or IL-22/nLNP groups did not exhibit any between-group compositional difference.

A donut graph was generated to visualize the microbial distribution within each group and identify the top seven most abundant phyla (FIG. 8C). Firmicutes and Bacteroidetes were dominant in the healthy mouse group, but Verrucomicrobia represented a large portion of the populations in all 2% DSS-treated groups. A Venn diagram was used to display the sharing of bacterial phyla between and across the groups. The total numbers of bacterial phyla in each group were as follows: healthy, 125; PBS, 110; blank nLNPs, 122; and IL-22/nLNPs, 129. Ninety-six phyla overlapped across all four groups (FIG. 8D). A heatmap and bar graph was also generated showing the relative microbial abundances and found that Bacteroidetes was the most abundant phylum in all groups, and there were more Firmicutes in the healthy group (FIG. 8E). To further investigate the top two most abundant phyla, Bacteroidetes (B) and Firmicutes (F), a side-by-side comparison was performed and the F to B ratio (F/B) was measured. The F/B was highest in the healthy group compared to the other groups (FIG. 8G-H). Distance matrix measurements between the groups were assessed using hierarchical clustering analysis, which revealed that Bacteriodetes and Firmicutes are relatively more abundant in all groups compared to other species (FIG. 8F). Collectively, the data demonstrate that oral administration of IL-22 mRNA-loaded nLNPs had no noticeable impact or apparent direct action on the gut microbiota composition.

### Discussion

Oral mRNA delivery is a promising yet understudied approach for treating inflammatory bowel disease (IBD). It was previously shown that orally delivered ginger-derived nanoparticles (GDNPs) targeted the colon and potently delivered CD-98 siRNA and small molecule drug candidate 6-shogaol to treat UC. It was also discovered that three types of lipids contribute more than 90% of the total lipids in GDNPs. These three lipids are phosphatidic acid (PA), monogalactosyldiacylglycerol (MGDG), and digalactosyldiacylglycerol (DGDG). It was hypothesized that mixing these three lipids at the ratio found in GDNPs would enable us to form nanoparticles that would retain the nanostructure of GDNPs and might prove useful as a well-defined nano-gene delivery system.

Here, these hypotheses were tested. It was found that a film hydration method similar to that used to make liposomal nanoparticles could be applied to self-organize a lipid mixture containing PA, MGDG, and DGDG into new lipid nanoparticles (nLNPs) with properties nearly identical to those of GDNPs (e.g., a spherical shape). It was then investigated the efficacy of oral delivery of IL-22 mRNA-loaded nLNPs as a novel therapeutic strategy for treating UC.

Inspired by the colon-targeting ability of ginger-derived nanoparticles (GDNPs), lipid nanoparticles that contain the three major lipids identified in GDNPs was reversely engineered. When mixed at the ratio found in GDNPs, the selected lipids (phosphatidic acid, monogalactosyldiacylglycerol, and digalactosyldiacylglycerol; 5:2:3) self-assembled into new lipid nanoparticles (nLNPs) in phosphate-buffered saline. The IL-22-mRNA was encapsulated within the nLNPs, as enhanced IL-22 expression in the colon is known to have potent anti-inflammatory efficacy against ulcerative colitis (UC). The IL-22 mRNA-loaded nLNPs (IL-22/nLNPs) were found to be about 200 nm in diameter and have a zeta potential of −18 mV. Oral delivery of IL-22/nLNPs elevated the protein expression level of IL-22 in the colonic mucosa of mice. In a mouse model of acute colitis, mice fed with IL-22/nLNPs experienced an accelerated healing process, as indicated by the recovery of more body weight and colon length as well as reduction of the histological index, colonic MPO activity, fecal lipocalin concentration, and mRNA expression levels of pro-inflammatory cytokines (TNF-α, IL-6, and IL-1 3). The results suggest that the reversely engineered nLNP is an excellent mRNA delivery platform for treating ulcerative colitis.

IL-22 primarily contributes to intestinal health by regulating epithelial homeostasis, which is established by building a tight epithelial barrier, regulating cell growth and permeability, and secreting mucus and antimicrobial proteins [20]. The IL-22-mediated production of mucin in mucosal tissues is the first line of defense against commensal bacteria and pathogens [21]. Specialized goblet cells generate the inner mucus layer in the colon to protect the host under normal conditions [22]. Disease-induced IL-22 deficiency can lead to inhibition of the goblet cell hyperplasia normally seen in response to an infection [23]. The presence of IL-22 and the production of membrane-bound mucins, such as Muc-1, Muc-3, and Muc-10, contribute to protecting the mucus layer from colitis-associated destruction and restoring goblet cells [24-26]. Recent investigations indicate that IL-22-mediated Muc-2 and Muc-3 expression helps alleviate DSS-induced colitis [27]. These studies suggest that lack of IL-22 may cause goblet cell deficiency and prevent healthy mucus layer development. Thus, it was speculated that targeted delivery of IL-22 to the colon might effectively improve mucosal wound healing following colitis. Sugimoto et al. [6] confirmed that local microinjection of IL-22 DNA to the mouse colon enhanced STAT3 activation within colonic epithelial cells and induced STAT3-dependent expression of mucus-associated molecules and reinstitution of mucus-producing goblet cells. However, it has been reported that long-term treatment with IL-22 or continuous activation of the IL-22 pathway may increase the risk of colitis-associated cancer [28, 29].

Recent decades have seen tremendous progress in the development of gene delivery techniques and their use in regulating disease-related proteins [30, 31]. Both mRNA and DNA have been widely studied for their ability to upregulate a target protein in vitro [32, 33]. Generally, DNA delivery is associated with delayed efficacy because the DNA must reach the nucleus to regulate protein expression. This nature of DNA is seen as a barrier to developing DNA-based therapeutics [34]. In contrast, an mRNA needs only to reach the cytoplasm, and thus its delivery can yield a more rapid increase in the target protein level [35]. Here, examined were both mRNA and pDNA for their ability to enhance the IL-22 protein level of cultured colonic epithelial cells. Consistent with the general thinking described above, it was found that mRNA delivery yielded more efficient IL-22 protein production compared to pDNA delivery. However, mRNAs are inherently unstable and vulnerable to degradation by ribonucleases. Thus, it is important to stabilize mRNAs for their in vivo delivery [36]. It is well known that modifying mRNA structural elements, particularly the 5′ cap, 5′ and 3′ untranslated regions (UTRs), coding region, and poly-A tail (which interacts with PBAPs to prevent mRNA degradation), can help reduce the excessive immunogenicity of an mRNA and improve its intracellular stability and translation efficiency [37]. A poly-A tail was added to the construct to improve the mRNA's stability. Purification of the mRNA is also necessary to ameliorate the instability and immunogenicity of mRNA [38]. As byproducts remaining in the IVT IL-22 mRNA solution may activate unexpected immune responses, the mRNA was purified by removing triphosphate, unreacted nucleotides, remaining enzymes, and residual salts.

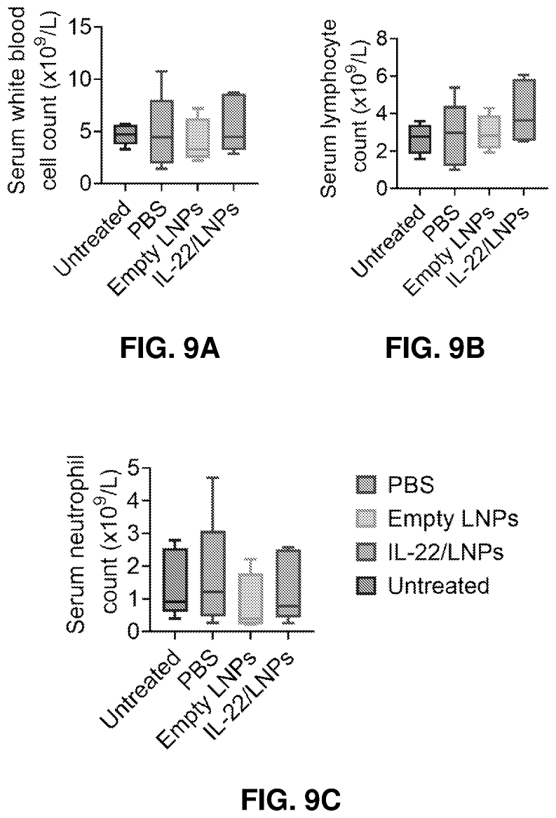

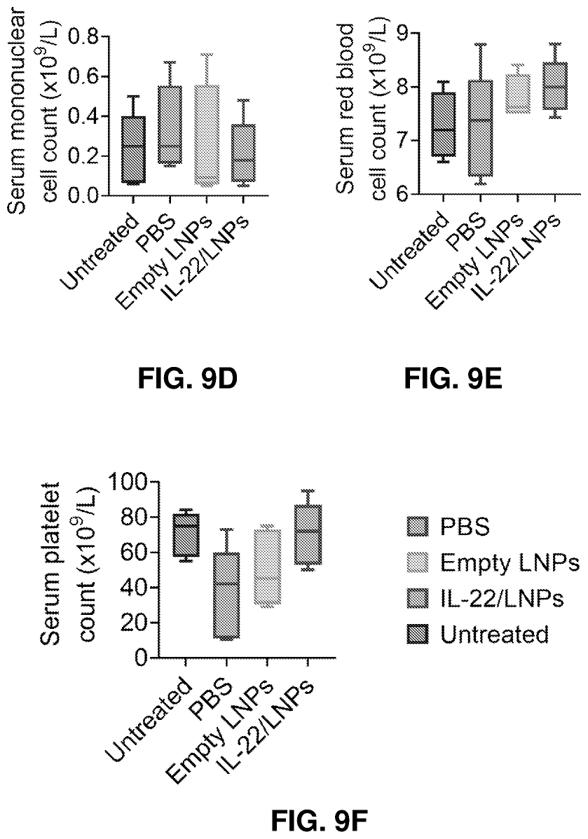

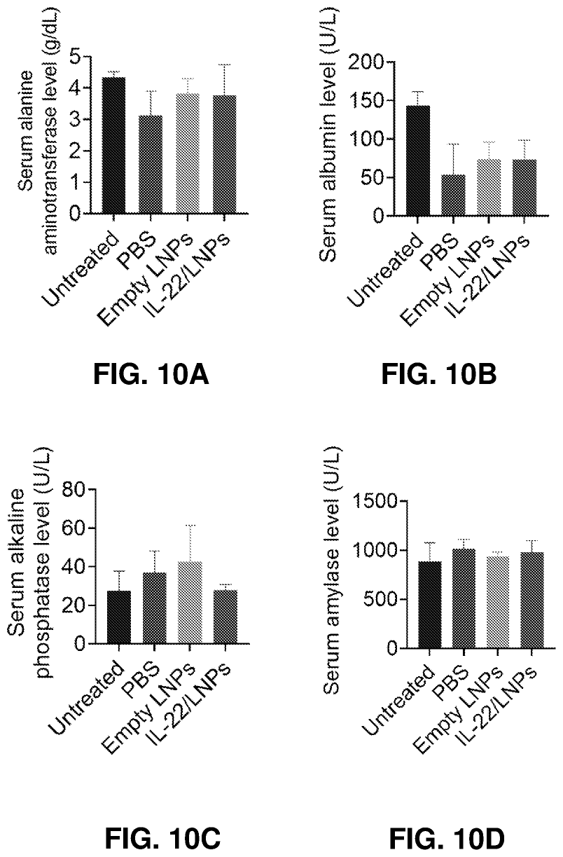

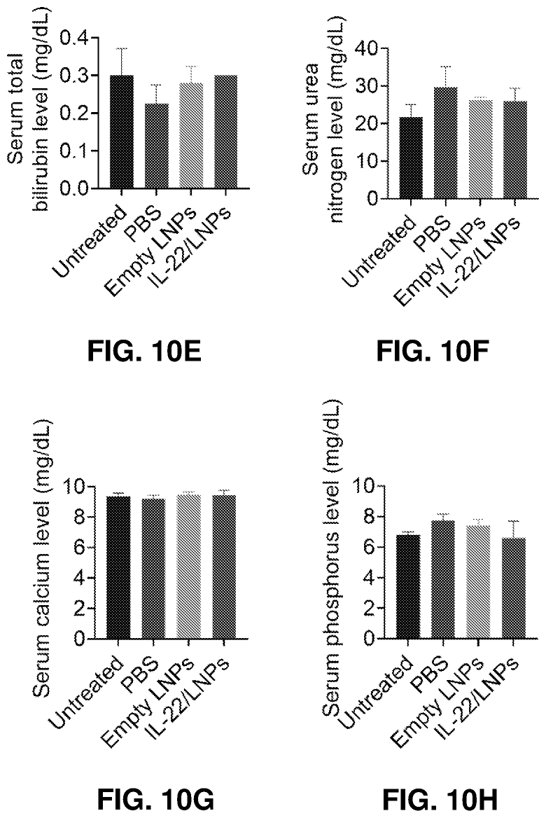

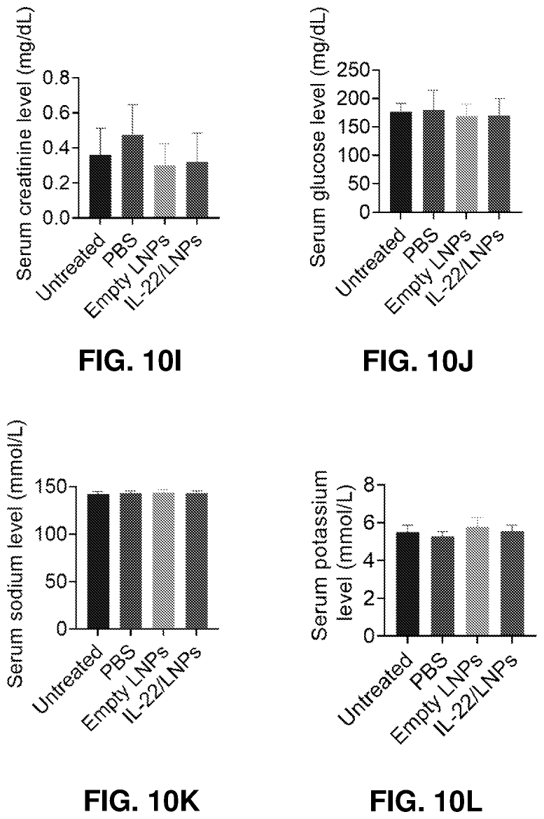

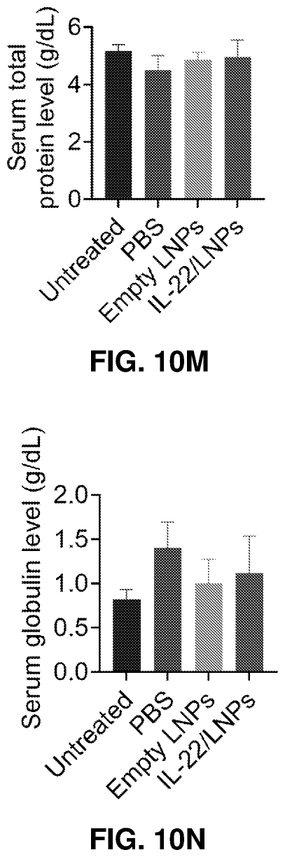

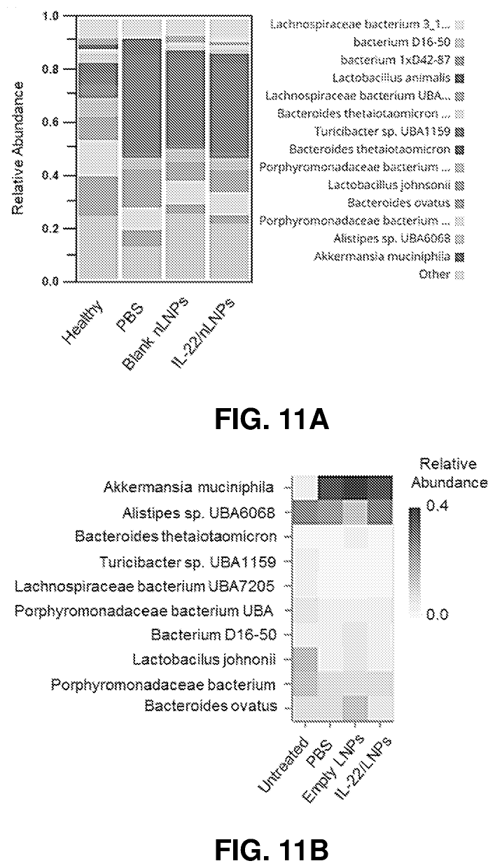

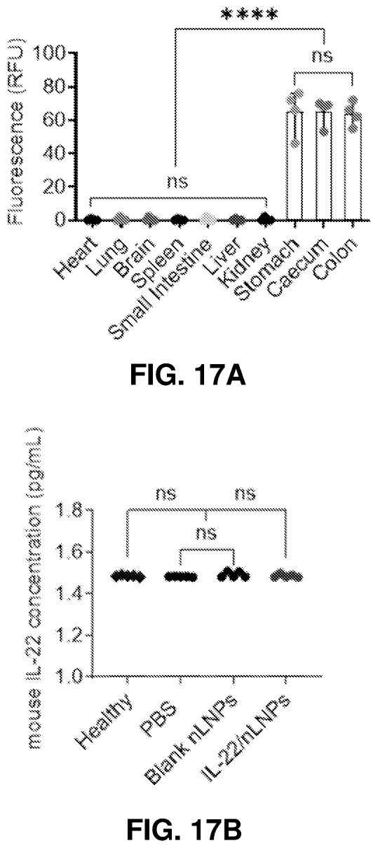

Subsequent toxicity results, including histology, spleen index, blood counting (FIG. 9), and blood chemistry (FIG. 10), demonstrated that the IL-22 mRNA did not generate any immune response associated with side effects. The biodistribution study also showed that orally delivered IL-22/nLNPs were restricted to the colon and stomach (a direct contact), with no observable distribution in other organs (FIG. 17A) and IL-22 protein expression was not increased in the blood (FIG. 17B). Such property of nLNPs-based oral gene delivery may largely improve the safety (reduce the systemic toxicity) of the treatment. However, the present work uses a non-modified IL-22 open reading frame. The translation efficiency (and protein expression) of IL-22 in the system could be improved by performing codon optimization of the open reading frame. Recently, codon-optimization of mRNAs has been exploited to impact the protein production rate [39]. For example, N1-methyl-pseudouridine nucleotide substitution was found to increase base pair stability and thereby improve mRNA translation [40].

At present, most gene formulations are delivered intravenously to increase their systemic bioavailability, and the therapeutics reach the target by either active targeting (e.g., antibody-guided targeting) or passive targeting (following the blood circulation). These gene delivery paths are indirect, and the therapeutics may cause unexpected side effects. Oral delivery is considered to be a better option, assuming that the colon-targeting formulation is sufficiently stable during GI tract transit. Having established that orally delivered GDNPs target the colon [41, 42], analyzed were the chemical components of GDNPs and set out to reverse engineer the nanostructure. LC-MS/MS-based lipid analysis revealed that GDNP lipids contain high percentages of the galactolipids, DGDG and MGDG, as well as the phospholipid, PA. The two galactolipids are commonly found in plant leaves, where they form the stable lipid bilayer structure of the chloroplast. Together, these three components make up more than 90% of the lipids isolated from GDNPs. Interestingly, many plant-derived nanoparticles (e.g., those from tea leaves, grape leaves, and ginger) lack cholesterol but are still sufficiently stable to target the lower part of the GI tract. Here, mixed were DGDG, MGDG, and PA at the ratio found in GDNPs, and found that this mixture successfully formed nanosized particles in PBS. To encapsulate the IL-22 mRNA, a cationic polymer was used to stabilize the mRNA and neutralize its native negative charge, which would block its encapsulation into the negatively charged reversely engineered nLNPs. The cationic polymer, turbofectamine, has two positively charged head groups connected by a 5-10 carbon/heteroatom linker and served as an anchor to stabilize the mRNA within the nLNPs. Nanocarriers such as nLNPs were generally internalized through endocytosis. Once they reach target cells, nLNPs might be internalized by multiple mechanisms, including macropinocytosis and clathrin- and/or caveolae-mediated endocytosis. The utilized endocytic pathway depends on the properties of the nanoparticle and the cell type. Endosomal escape is crucial for effective mRNA delivery. Although the underlying mechanism has not yet been fully elucidated, positively charged lipids such as Turbofectamine may facilitate electrostatic interaction and fusion with negatively charged endosomal membranes, enabling mRNA molecules to leak into the cytoplasm.

Synthetic nanoparticles offer a more consistent end product compared to their naturally derived counterparts and have been applied in clinical studies. For example, lipid nanoparticles (LNPs) have been used to successfully deliver mRNAs to prevent the spread of SARS-CoV-2 [43, 44]. Due to the rise of mRNA therapeutics, the pharmaceutical industry has faced a vast paradigm shift in developing this new class of gene therapeutics and updating drug development plans from focusing on small molecules to emphasizing gene-based drugs [45-48]. Indeed, gene-based biopharmaceuticals have shown remarkable benefits and efficacy over traditional chemical drugs, with far fewer adverse effects and higher specificity [49, 50]. The advantage of using well-defined reverse-engineered nLNPs as a gene-drug delivery system, compared to the original plant-derived nanoparticles, is that the nLNPs have a consistent chemical composition and characteristics, with a reduced batch-to-batch variation. In addition, the reverse-engineered nLNPs essentially inherit the original galactolipid/phosphoric lipid ratio, lipid bilayer structure, and colon-targeting of GDNPs. Because DGDG, MGDG, and PA are abundant in many green leafy plants [51-53], it might be feasible to mass-produce nLNPs from these three lipids.

Here, it was shown that the reversely engineered nLNPs could be embodied as a robust drug delivery system that targets encapsulated IL-22 mRNAs to the colon tissue and induces colonic IL-22 protein expression. It is believed that the targeted cells are most likely to be the intestinal epithelial cells. However, further experiments will be needed to rule out the possibility that other cells, such as macrophages, could take up the IL-22 mRNA and express the observed colonic IL-22 protein after the oral administration of IL-22 mRNA-loaded lipid nanoparticles.

This study demonstrates that oral delivery of IL-22/nLNPs can target the injured intestinal mucosa and accelerate the healing phase in a mouse model of intestinal wound healing. The results demonstrate that orally delivered IL-22/nLNPs induce colonic IL-22 protein expression and might accelerate the restitution of intestinal barrier function, as shown by the histological results (FIG. 7A-D). These findings agree with previous studies showing that IL-22 regulates the homeostasis of the intestinal epithelium and is critical for the regeneration of the intestine under inflammation [54]. It is suggested that the IL-22-mediated reduction of pro-inflammatory cytokines, such as TNF-α, IL-1β, IL-6, and the mucosal infiltration of neutrophils, as observed in the experiments (FIG. 6) may be conducive to the restitution of the intestinal barrier function. Interestingly, it was found that orally administrated IL-22/nLNPs had no direct action on the gut microbiota composition and that the IL-22 mRNA treatment appears to target the colonic mucosa.

### CONCLUSIONS

Herein described is a novel lipid nanoparticle-based delivery method for targeting IVT-generated IL-22 mRNA to the colon. The lipid nanoparticles were formed using the major lipids from GDNPs (DGDG, MGDG, and PA) at the ratio found in these natural nanoparticles. Oral delivery of IL-22/nLNPs yielded enhanced IL-22 protein expression in the colonic mucosa of mice. IL-22 mRNA delivery also reduced inflammatory parameters (body weight/colon length changes and the upregulations of fecal MPO [representing the mucosal infiltration of neutrophils], Lcn-2, and pro-inflammatory cytokines in intestinal epithelial cells and the mucus layer) without affecting the microbial composition in the gut. The results revealed that the dynamic alteration of IL-22 is important for maintaining intestinal homeostasis during inflammation, providing new insight into the role of IL-22 in regulating the homeostasis of the intestinal epithelium during inflammation. Whereas long-term treatment with IL-22 has been suggested to increase the risk of colitis-associated cancer [29], the current study suggested that a short-term targeted administration of IL-22 mRNA to the colonic mucosa may be beneficial in treating UC. In sum, using the precisely fabricated lipid mRNA delivery system, it was demonstrated that oral delivery of IL-22/nLNPs may provide a potent gene therapy strategy for the treatment of ulcerative colitis.

### REFERENCES

[1] S. Alatab, S. G. Sepanlou, K. Ikuta, H. Vahedi, C. Bisignano, S. Safiri, A. Sadeghi, M. R. Nixon, A. Abdoli, H. Abolhassani, V. Alipour, M. A. H. Almadi, A. Almasi-Hashiani, A. Anushiravani, J. Arabloo, S. Atique, A. Awasthi, A. Badawi, A. A. A. Baig, N. Bhala, A. Bijani, A. Biondi, A. M. Borzí, K. E. Burke, F. Carvalho, A. Daryani, M. Dubey, A. Eftekhari, E. Fernandes, J. C. Fernandes, F. Fischer, A. Haj-Mirzaian, A. Haj-Mirzaian, A. Hasanzadeh, M. Hashemian, S. I. Hay, C. L. Hoang, M. Househ, O. S. Ilesanmi, N. Jafari Balalami, S. L. James, A. P. Kengne, M. M. Malekzadeh, S. Merat, T. J. Meretoja, T. Mestrovic, E. M. Mirrakhimov, H. Mirzaei, K. A. Mohammad, A. H. Mokdad, L. Monasta, I. Negoi, T. H. Nguyen, C. T. Nguyen, A. Pourshams, H. Poustchi, M. Rabiee, N. Rabiee, K. Ramezanzadeh, D. L. Rawaf, S. Rawaf, N. Rezaei, S. R. Robinson, L. Ronfani, S. Saxena, M. Sepehrimanesh, M. A. Shaikh, Z. Sharafi, M. Sharif, S. Siabani, A. R. Sima, J. A. Singh, A. Soheili, R. Sotoudehmanesh, H. A. R. Suleria, B. E. Tesfay, B. Tran, D. Tsoi, M. Vacante, A. B. Wondmieneh, A. Zarghi, Z.-J. Zhang, M. Dirac, R. Malekzadeh, M. Naghavi, The global, regional, and national burden of inflammatory bowel disease in 195 countries and territories, 1990-2017: a systematic analysis for the Global Burden of Disease Study 2017, The Lancet Gastroenterology & Hepatology 5(1) (2020) 17-30.
[2] C. Fiocchi, Inflammatory bowel disease pathogenesis: where are we?, J Gastroenterol Hepatol 30 Suppl 1 (2015) 12-8.
[3] R. J. Xavier, D. K. Podolsky, Unravelling the pathogenesis of inflammatory bowel disease, Nature 448(7152) (2007) 427-34.
[4] Y. Z. Zhang, Y. Y. Li, Inflammatory bowel disease: pathogenesis, World J Gastroenterol 20(1) (2014) 91-9.
[5] M. F. Neurath, Current and emerging therapeutic targets for IBD, Nat Rev Gastroenterol Hepatol 14(5) (2017) 269-278.
[6] K. Sugimoto, A. Ogawa, E. Mizoguchi, Y. Shimomura, A. Andoh, A. K. Bhan, R. S. Blumberg, R. J. Xavier, A. Mizoguchi, IL-22 ameliorates intestinal inflammation in a mouse model of ulcerative colitis, J Clin Invest 118(2) (2008) 534-44.
[7] J. Sung, C. Yang, E. Viennois, M. Zhang, D. Merlin, Isolation, Purification, and Characterization of Ginger-derived Nanoparticles (GDNPs) from Ginger, Rhizome of Zingiber officinale, Bio Protoc 9(19) (2019).
[8] J. Sung, C. Yang, J. F. Collins, D. Merlin, Preparation and Characterization of Ginger Lipid-derived Nanoparticles for Colon-targeted siRNA Delivery, Bio Protoc 10(14) (2020).
[9] D. Long, C. Yang, J. Sung, D. Merlin, Atomic Force Microscopy to Characterize Ginger Lipid-Derived Nanoparticles (GLDNP), Bio Protoc 11(7) (2021) e3969.
[10] R. Gelder, M. Zastrow, A. Yool, W. Dement, J. Barchas, J. Eberwine, Amplified RNA synthesized from limited quantities of heterogeneous cDNA, Biochemistry 87 (1990) 1663-1667.
[11] G. D. R., The cap and poly(A) tail function synergistically to regulate mRNA translational efficiency, Genes & development 5 (1991) 2108-2116.
[12] A. Puri, K. Loomis, B. Smith, J. Lee, A. Yavlovich, E. Heldman, R. Blumenthal, Lipid-Based Nanoparticles as Pharmaceutical Drug Carriers: From Concepts to Clinic, Critical Review of Therapeutic Drug Carrier System 26 (2009) 523-580.
└13┘ Y. Zhao, L. Huang, Lipid nanoparticles for gene delivery, Advances in genetics 88 (2014) 13-36.
[14] M. Zhang, X. Wang, M. K. Han, J. F. Collins, D. Merlin, Oral administration of ginger-derived nanolipids loaded with siRNA as a novel approach for efficient siRNA drug delivery to treat ulcerative colitis, Nanomedicine (Lond) 12(16) (2017) 1927-1943.
[15] M. Zhang, E. Viennois, M. Prasad, Y. Zhang, L. Wang, Z. Zhang, M. K. Han, B. Xiao, C. Xu, S. Srinivasan, D. Merlin, Edible ginger-derived nanoparticles: A novel therapeutic approach for the prevention and treatment of inflammatory bowel disease and colitis-associated cancer, Biomaterials 101 (2016) 321-40.
[16] B. Xiao, Q. Chen, Z. Zhang, L. Wang, Y. Kang, T. Denning, D. Merlin, TNFalpha gene silencing mediated by orally targeted nanoparticles combined with interleukin-22 for synergistic combination therapy of ulcerative colitis, J Control Release 287 (2018) 235-246.
[17] H. Laroui, S. A. Ingersoll, H. C. Liu, M. T. Baker, S. Ayyadurai, M. A. Charania, F. Laroui, Y. Yan, S. V. Sitaraman, D. Merlin, Dextran sodium sulfate (DSS) induces colitis in mice by forming nano-lipocomplexes with medium-chain-length fatty acids in the colon, PLoS One 7(3) (2012) e32084.
[18] D. A. Williams, Inflammatory cytokines and mucosal injury, J Natl Cancer Inst Monogr (29) (2001) 26-30.
[19] S. Nikolaus, J. Bauditz, P. Gionchetti, C. Witt, H. Lochs, S. Schreiber, Increased secretion of pro-inflammatory cytokines by circulating polymorphonuclear neutrophils and regulation by interleukin 10 during intestinal inflammation, Gut 42(4) (1998) 470-6.
[20] M. Keir, Y. Yi, T. Lu, N. Ghilardi, The role of IL-22 in intestinal health and disease, J Exp Med 217(3) (2020) e20192195.
[21] M. E. Johansson, G. C. Hansson, Immunological aspects of intestinal mucus and mucins, Nat Rev Immunol 16(10) (2016) 639-49.
[22] A. Ermund, A. Schutte, M. E. Johansson, J. K. Gustafsson, G. C. Hansson, Studies of mucus in mouse stomach, small intestine, and colon. I. Gastrointestinal mucus layers have different properties depending on location as well as over the Peyer's patches, Am J Physiol Gastrointest Liver Physiol 305(5) (2013) G341-7.
[23] J. E. Turner, B. Stockinger, H. Helmby, IL-22 mediates goblet cell hyperplasia and worm expulsion in intestinal helminth infection, PLoS Pathog 9(10) (2013) e1003698.
[24] T. Shirazi, R. Longman, A. Corfield, C. Probert, Mucins and inflammatory bowel disease, Postgraduate Medical Journal 76 (2000) 473-478.
[25] S. B. Ho, L. A. Dvorak, R. E. Moor, A. C. Jacobson, M. R. Frey, J. Corredor, D. B. Polk, L. L. Shekels, Cysteine-rich domains of muc3 intestinal mucin promote cell migration, inhibit apoptosis, and accelerate wound healing, Gastroenterology 131(5) (2006) 1501-17.
[26] A. Corfield, D. Carroll, N. Myerscough, C. Probert, MUCINS IN THE GASTROINTESTINAL TRACT IN HEALTH AND DISEASE, Frontiers in Bioscience 6 (2001).
[27] M. Van der Sluis, B. A. De Koning, A. C. De Bruijn, A. Velcich, J. P. Meijerink, J. B. Van Goudoever, H. A. Buller, J. Dekker, I. Van Seuningen, I. B. Renes, A. W. Einerhand, Muc2-deficient mice spontaneously develop colitis, indicating that MUC2 is critical for colonic protection, Gastroenterology 131(1) (2006) 117-29.
[28] T. Arshad, F. Mansur, R. Palek, S. Manzoor, V. Liska, A Double Edged Sword Role of Interleukin-22 in Wound Healing and Tissue Regeneration, Front Immunol 11 (2020) 2148.
[29] A. Mizoguchi, A. Yano, H. Himuro, Y. Ezaki, T. Sadanaga, E. Mizoguchi, Clinical importance of IL-22 cascade in IBD, J Gastroenterol 53(4) (2018) 465-474.
[30] S. Nimesh, S. Halappanavar, N. K. Kaushik, P. Kumar, Advances in Gene Delivery Systems, Biomed Res Int 2015 (2015).
[31] S. Goverdhana, M. Puntel, W. Xiong, J. M. Zirger, C. Barcia, J. F. Curtin, E. B. Soffer, S. Mondkar, G. D. King, J. Hu, S. A. Sciascia, M. Candolfi, D. S. Greengold, P. R. Lowenstein, M. G. Castro, Regulatable gene expression systems for gene therapy applications: progress and future challenges, Mol Ther 12(2) (2005) 189-211.
[32] N. Veiga, M. Goldsmith, Y. Granot, D. Rosenblum, N. Dammes, R. Kedmi, S. Ramishetti, D. Peer, Cell specific delivery of modified mRNA expressing therapeutic proteins to leukocytes, Nat Commun 9(1) (2018) 4493.
[33] J. Buck, P. Grossen, P. R. Cullis, J. Huwyler, D. Witzigmann, Lipid-Based DNA Therapeutics: Hallmarks of Non-Viral Gene Delivery, ACS Nano 13(4) (2019) 3754-3782.
[34] A. J. Phillips, The challenge of gene therapy and DNA delivery, J Pharm Pharmacol 53(9) (2001) 1169-74.
[35] A. Wadhwa, A. Aljabbari, A. Lokras, C. Foged, A. Thakur, Opportunities and Challenges in the Delivery of mRNA-based Vaccines, Pharmaceutics 12(2) (2020).
[36] S. Guan, J. Rosenecker, Nanotechnologies in delivery of mRNA therapeutics using nonviral vector-based delivery systems, Gene Ther 24(3) (2017) 133-143.
[37] M. Z. Wu, H. Asahara, G. Tzertzinis, B. Roy, Synthesis of low immunogenicity RNA with high-temperature in vitro transcription, RNA 26(3) (2020) 345-360.
[38] M. Baiersdorfer, G. Boros, H. Muramatsu, A. Mahiny, I. Vlatkovic, U. Sahin, K. Kariko, A Facile Method for the Removal of dsRNA Contaminant from In Vitro-Transcribed mRNA, Mol Ther Nucleic Acids 15 (2019) 26-35.
[39] V. P. Mauro, Codon Optimization in the Production of Recombinant Biotherapeutics: Potential Risks and Considerations, BioDrugs 32(1) (2018) 69-81.
[40] D. M. Mauger, B. J. Cabral, V. Presnyak, S. V. Su, D. W. Reid, B. Goodman, K. Link, N. Khatwani, J. Reynders, M. J. Moore, I. J. McFadyen, mRNA structure regulates protein expression through changes in functional half-life, Proc Natl Acad Sci USA 116(48) (2019) 24075-24083.
[41] M. Zhang, D. Merlin, Nanoparticle-Based Oral Drug Delivery Systems Targeting the Colon for Treatment of Ulcerative Colitis, Inflamm Bowel Dis 24(7) (2018) 1401-1415.
[42] C. Yang, D. Long, J. Sung, Z. Alghoul, D. Merlin, Orally Administered Natural Lipid Nanoparticle-Loaded 6-Shogaol Shapes the Anti-Inflammatory Microbiota and Metabolome, Pharmaceutics 13(9) (2021).
[43] X. Hou, T. Zaks, R. Langer, Y. Dong, Lipid nanoparticles for mRNA delivery, Nat Rev Mater (2021) 1-17.
[44] A. Khurana, P. Allawadhi, I. Khurana, S. Allwadhi, R. Weiskirchen, A. K. Banothu, D. Chhabra, K. Joshi, K. K. Bharani, Role of nanotechnology behind the success of mRNA vaccines for COVID-19, Nano Today 38 (2021) 101142.
[45] M. Foldvari, D. W. Chen, N. Nafissi, D. Calderon, L. Narsineni, A. Rafiee, Non-viral gene therapy: Gains and challenges of non-invasive administration methods, J Control Release 240 (2016) 165-190.
[46] S. Sripriyalakshmi, P. Jose, A. Ravindran, C. H. Anjali, Recent trends in drug delivery system using protein nanoparticles, Cell Biochem Biophys 70(1) (2014) 17-26.
[47] M. Wang, Y. Yu, C. Liang, A. Lu, G. Zhang, Recent Advances in Developing Small Molecules Targeting Nucleic Acid, Int J Mol Sci 17(6) (2016).
[48] H. Yin, K. J. Kauffman, D. G. Anderson, Delivery technologies for genome editing, Nat Rev Drug Discov 16(6) (2017) 387-399.
[49] U. Sahin, K. Kariko, O. Tureci, mRNA-based therapeutics—developing a new class of drugs, Nat Rev Drug Discov 13(10) (2014) 759-80.
[50] L. Zhong, Y. Li, L. Xiong, W. Wang, M. Wu, T. Yuan, W. Yang, C. Tian, Z. Miao, T. Wang, S. Yang, Small molecules in targeted cancer therapy: advances, challenges, and future perspectives, Signal Transduct Target Ther 6(1) (2021) 201.
[51] R. Cook, J. Lupette, C. Benning, The Role of Chloroplast Membrane Lipid Metabolism in Plant Environmental Responses, Cells 10(3) (2021).
[52] C. W. Yu, Y. T. Lin, H. M. Li, Increased ratio of galactolipid MGDG:DGDG induces jasmonic acid overproduction and changes chloroplast shape, New Phytol 228(4) (2020) 1327-1335.
[53] E. Reszczynska, A. Hanaka, Lipids Composition in Plant Membranes, Cell Biochem Biophys 78(4) (2020) 401-414.
[54] X. Zhang, S. Liu, Y. Wang, H. Hu, L. Li, Y. Wu, D. Cao, Y. Cai, J. Zhang, X. Zhang, Interleukin22 regulates the homeostasis of the intestinal epithelium during inflammation, Int J Mol Med 43(4) (2019) 1657-1668.

It is understood that the disclosed method and compositions are not limited to the particular methodology, protocols, and reagents described as these may vary. It is also to be understood that the terminology used herein is for the purpose of describing particular embodiments only and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, reference to “a nanoparticle” includes a plurality of such nanoparticles, reference to “the nanoparticle” is a reference to one or more nanoparticles and equivalents thereof known to those skilled in the art, and so forth.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. may include numerous options and alternatives, this should not be construed as, and is not an admission that, such options and alternatives are equivalent to each other or, in particular, are obvious alternatives.

Every compound disclosed herein is intended to be and should be considered to be specifically disclosed herein. Further, every subgroup that can be identified within this disclosure is intended to be and should be considered to be specifically disclosed herein. As a result, it is specifically contemplated that any compound, or subgroup of compounds can be either specifically included for or excluded from use or included in or excluded from a list of compounds.

Those skilled in the art will recognize or be able to ascertain using no more than routine experimentation many equivalents to the specific embodiments of the method and compositions described herein. Such equivalents are intended to be encompassed by the following claims.

1. A lipid nanoparticle comprising phosphatidic acid (PA), monogalactosyldiacylglycerol (MGDG), and digalactosyldiacylglycerol (DGDG), wherein the PA, MGDG, and DGDG are present in the nanoparticle in a ratio of 3 to 7, 1 to 3, and 2 to 4, respectively, and wherein the PA, MGDG, and DGDG make up 90% or more of the total lipid in the nanoparticle.

2. The lipid nanoparticle of claim 1, wherein the PA, MGDG, and DGDG are each of high purity before being mixed to form the nanoparticle;
wherein the PA, MGDG, and DGDG are each of at least 97% purity before being mixed to form the nanoparticle; or
wherein the PA, MGDG, and DGDG are each of at least 98% purity before being mixed to form the nanoparticle; or
wherein the PA, MGDG, and DGDG are each of at least 99% purity before being mixed to form the nanoparticle.

3. The lipid nanoparticle of claim 1, wherein no component of the nanoparticle is obtained from ginger.

4. The lipid nanoparticle of claim 1, wherein the PA, MGDG, and DGDG are present in the nanoparticle in a ratio of 5:2:3,
wherein the PA, MGDG, and DGDG make up 95% or more of the total lipid in the nanoparticle; or
wherein the PA, MGDG, and DGDG make up 97% or more of the total lipid in the nanoparticle; or
wherein the PA, MGDG, and DGDG make up 99% or more of the total lipid in the nanoparticle.

5-10. (canceled)

11. The lipid nanoparticle of claim 1, further comprising ionizable lipids, wherein the ionizable lipids make up 10% or less of the total lipid in the nanoparticle, optionally wherein the ionizable lipids comprise cationic lipids.

12. (canceled)

13. The lipid nanoparticle of claim 1, further comprising one or more surface modifications.

14. The lipid nanoparticle of claim 1, further comprising one or more payload components;
wherein at least one of the payload components is a compound for delivery to intestine.

15. (canceled)

16. The lipid nanoparticle of claim 14, wherein at least one of the payload components is a therapeutic agent for treatment of one or more of inflammatory bowel disease, ulcerative colitis, and Chron's disease.

17-18. (canceled)

19. The lipid nanoparticle of claim 14, wherein at least one of the payload components is a therapeutic agent for treatment of cancer, optionally wherein the therapeutic agent is for the treatment of colon cancer.

20. (canceled)

21. The lipid nanoparticle of claim 14, wherein at least one of the payload components is a therapeutic agent for treatment of coronavirus infection.

22. The lipid nanoparticle of claim 14, wherein at least one of the payload components is RNA, DNA, and/or protein;
optionally wherein at least one of the payload components is pDNA.

23. The lipid nanoparticle of claim 14, wherein at least one of the payload components is mRNA, or siRNA or a replicate for siRNA,
optionally wherein at least one of the payload components is an mRNA encoding IL-22.

24-28. (canceled)

29. A pharmaceutical composition comprising the lipid nanoparticle of claim 1, optionally wherein the composition is formulated for oral administration.

30. (canceled)

31. A method comprising administering the composition of claim 29 to a subject.

32. The method of claim 31, wherein the composition is administered orally to the subject.

33. The method of claim 31, wherein the subject is suffering a disease or condition, optionally wherein the subject is suffering from inflammatory bowel disease, ulcerative colitis, or Crohn's disease.

34-36. (canceled)

37. The method of claim 31, wherein the subject is suffering cancer, optionally wherein the subject is suffering from colon cancer.

38. (canceled)

39. The method of claim 31, wherein the subject is suffering coronavirus infection.

40-42. (canceled)

43. A drug delivery system for delivery of a drug, the drug delivery system comprising the lipid nanoparticle of claim 1.

44. The drug delivery system of claim 43, wherein the drug delivery system is formulated for oral delivery, intravascular delivery, or intramuscular delivery.

45-46. (canceled)
